(19) United States
(12) Patent Application Publication
(10) Pub. No.: US 2025/0133277 A1
(43) Pub. Date: Apr. 24, 2025

(54) MULTISPECTRAL FILTER ARRAYS FOR STEREOSCOPIC CAMERAS

(71) Applicant: Cilag GmbH International, Zug (CH)

(72) Inventors: Vanessa Bell, Dedham, MA (US); Steen Hansen, Copenhagen (DK); Jeremiah D. Henley, Fair Oaks, CA (US); Marco Kristensen, Rochester, NY (US)

(73) Assignee: Cilag GmbH International, Zug (CH)

(21) Appl. No.: 18/491,498

(22) Filed: Oct. 20, 2023

(51) Int. Cl. H04N 23/16; G06T 17/00; H04N 13/239; H04N 13/254; H04N 13/257; H04N 23/50; H04N 25/131

(52) U.S. Cl. CPC H04N 23/16; G06T 17/00; H04N 13/239; H04N 13/254; H04N 13/257; H04N 23/555; H04N 25/131

(57) ABSTRACT

Systems for stereoscopic visualization with multispectral filter arrays configured for capturing color imaging data and spectral imaging data. A system includes an emitter comprising a plurality of sources of electromagnetic radiation, including a visible source, a first spectral source that emits electromagnetic radiation within a first spectral waveband, and a second spectral source that emits electromagnetic radiation within a second spectral waveband. The system includes a first image sensor comprising a first multispectral filter array, wherein at least a portion of the first multispectral filter array transmits reflected electromagnetic radiation within the first spectral waveband. The system includes a second image sensor comprising a second multispectral filter array, wherein at least a portion of the second multispectral filter array transmits reflected electromagnetic radiation within the second spectral waveband. The system is such that the first spectral waveband is different from the second spectral waveband.

### TECHNICAL FIELD

This disclosure is directed to advanced visualization and digital imaging systems and methods and, more particularly but not entirely, to multispectral filter arrays for stereoscopic cameras.

### BACKGROUND

Endoscopic surgical instruments are often preferred over traditional open surgical devices because the small incision tends to reduce post-operative recovery time and associated complications. In some instances of endoscopic visualization, it is desirable to view a space with high-definition color imaging and further with one or more advanced visualization techniques providing additional information that cannot be discerned with the human eye. In many cases, and particularly when image data is utilized by a robotic surgical system, it is desirable to extract dimensional information from the scene using stereoscopic imaging, laser mapping, or some other means. However, these advanced visualization techniques require specialized components, and the space-constrained environment of an endoscope introduces numerous technical challenges when seeking to capture advanced visualization data of a surgical scene.

Spectral visualization data, including multispectral visualization data and fluorescence visualization data, can provide valuable information that aids in identifying certain tissue structures, chemical processes, reagents, biological processes, and tissue abnormalities. However, it is difficult to capture color visualization data and spectral data.

There are numerous endoscopic visualization systems seeking to capture advanced visualization data, such as multispectral data, fluorescence data, and dimensional information, while working within the space constrained environment of an endoscope. However, these systems do not address the technical challenges associated with capturing color imaging data, spectral imaging data, and dimensional information with an image sensor. It can be particularly difficult to capture each of the aforementioned visualization types when limited by the frame rate capabilities of an image sensor and/or the image sensor's relative inefficiencies at detecting different wavebands of electromagnetic radiation.

For example, commonly owned U.S. Patent Application Publication No. 2020/0404131, entitled “HYPERSPECTRAL AND FLUORESCENCE IMAGING WITH TOPOLOGY LASER SCANNING IN A LIGHT DEFICIENT ENVIRONMENT,” filed on Oct. 24, 2019, which is incorporated by reference in its entirety, describes an endoscopic visualization system for color and “specialty” imaging. In this disclosure, an emitter is configured to emit electromagnetic energy in wavelength bands within the visible spectrum, including red, green, and blue emissions, as well as specialty emissions, wherein the specialty emissions may include hyperspectral, fluorescence, or laser mapping emissions of electromagnetic energy. However, this disclosure does not describe a system for stereoscopic visualization including multispectral filter arrays for simultaneously capturing color imaging data and spectral imaging data.

In view of the foregoing, disclosed herein are systems, methods, and devices for generating advanced visualization video streams based on data output by a stereoscopic camera capable of outputting color imaging data, spectral imaging data, and dimensional information.

### BRIEF DESCRIPTIONS OF THE DRAWINGS

Non-limiting and non-exhaustive implementations of the disclosure are described with reference to the following figures, wherein like reference numerals refer to like parts throughout the various views unless otherwise specified. Advantages of the disclosure will become better understood with regard to the following description and accompanying drawings where:

FIG. 1A is a schematic illustration of an example system for endoscopic visualization with color imaging and advanced imaging;

FIG. 1B is a schematic illustration of an example image pickup portion of a system for endoscopic visualization with color imaging and advanced imaging;

FIG. 1C is a schematic illustration of an example emitter and controller of a system for endoscopic visualization with color imaging and advanced imaging;

FIG. 2A is a schematic block diagram of an example data flow for a time-sequenced visualization system;

FIG. 2B is a schematic block diagram of an example data flow for a time-sequenced visualization system;

FIG. 2C is a schematic flow chart diagram of a data flow for capturing and reading out data for a time-sequenced visualization system;

FIG. 3A is a schematic block diagram of an example system for processing data output by an image sensor with a controller in communication with an emitter and the image sensor;

FIG. 3B is a schematic block diagram of an example system for processing data output by an image sensor to generate color imaging data and advanced imaging data;

FIG. 3C is a schematic block diagram of an example system for processing data through a memory buffer to provide data frames to an image signal processor at regular intervals;

FIG. 4 is a schematic diagram of an illumination system for illuminating a light deficient environment according to a variable pulse cycle;

FIG. 5A is a schematic illustration of a Bayer pattern color filter array for an image sensor;

FIG. 5B is a schematic illustration of a quad Bayer pattern color filter array for an image sensor;

FIG. 6 is a schematic illustration of a multispectral filter array that may be applied to a single image sensor to output red-green-blue image data and spectral image data;

FIG. 7A is a schematic illustration of a multispectral filter array to be applied to a left channel image sensor in a stereoscopic camera;

FIG. 7B is a schematic illustration of a multispectral filter array to be applied to a right channel image sensor in a stereoscopic camera;

FIG. 8A is a schematic illustration of a multispectral filter array to be applied to a left channel image sensor in a stereoscopic camera;

FIG. 8B is a schematic illustration of a multispectral filter array to be applied to a right channel image sensor in a stereoscopic camera;

FIG. 9 is a schematic block diagram of a process flow for providing data to a controller to render a video stream output;

FIG. 10 illustrates a portion of the electromagnetic spectrum divided into a plurality of different wavebands which may be pulsed by sources of electromagnetic radiation of an emitter;

FIG. 11 is a schematic diagram illustrating a timing sequence for emission and readout for generating data frames in response to pulses of electromagnetic radiation; and

FIG. 12 is a schematic block diagram of an example computing device.

### DETAILED DESCRIPTION

Disclosed herein are systems, methods, and devices for digital visualization that may be primarily suited to medical applications such as medical endoscopic imaging. An embodiment of the disclosure is an endoscopic system for color visualization and “advanced visualization” of a scene. The advanced visualization includes one or more of multispectral imaging, fluorescence imaging, or topographical mapping. Data retrieved from the advanced visualization may be processed by one or more algorithms configured to determine characteristics of the scene. The advanced visualization data may specifically be used to identify tissue structures within a scene, estimate tissue dimensions and parameters, generate a three-dimensional topographical map of the scene, calculate dimensions of objects within the scene, identify margins and boundaries of different tissue types, and so forth.

Specifically disclosed herein are multispectral filter array (MSFA) designs for a stereoscopic camera comprising at least two image sensors. As described herein, an endoscopic visualization system is equipped with two or more image sensors for capturing stereoscopic visualization data. Each of the two or more image sensors may be equipped with a different MSFA configured to transmit electromagnetic radiation (EMR) to a pixel array. Each of the different MSFAs include red, green, and blue filters, and additionally include specialized spectral filters configured to transmit selected wavebands of EMR. The different MSFAs may be equipped with different spectral filters such that the two or more image sensors are configured to capture different types of spectral visualization data.

An embodiment of the disclosure is an endoscopic visualization system that includes an emitter, an image sensor, and a controller. The emitter includes a plurality of separate and independently actuatable sources of EMR that may be separately cycled on and off to illuminate a scene with pulses of EMR. The image sensor accumulates photons and converts this reading to an electrical charge. The image sensor reads out the electrical charge data to generate a plurality of data frames. The controller synchronizes operations of the emitter and the image sensor to output a desired visualization scheme based on user input, which may be provided via a surgical display system. The visualization scheme may include a selection of one or more of color imaging, multispectral imaging, fluorescence imaging, topographical mapping, or anatomical measurement.

In some implementations of the system, the controller instructs the emitter and the image sensor to operate in a synchronized sequence to output a video stream that includes one or more types of visualization (i.e., color imaging, multispectral imaging, fluorescence imaging, topographical mapping, or anatomical measurement). The controller instructs the emitter to actuate one or more of the plurality of EMR sources to pulse according to a variable pulse cycle. The controller instructs the image sensor to accumulate EMR and read out data according to a variable sensor cycle that is synchronized in time with the variable pulse cycle. The synchronized sequence of the emitter and the image sensor enables the image sensor to read out data corresponding with a plurality of different visualization types. For example, the image sensor may read out a color frame in response to the emitter pulsing a white light or other visible EMR, the image sensor may readout a multispectral frame in response to the emitter pulsing a multispectral waveband of EMR, the image sensor may read out data for calculating a three-dimensional topographical map in response to the emitter pulsing EMR in a mapping pattern, and so forth.

The systems, methods, and devices described herein are implemented for color visualization and advanced visualization. The advanced visualization techniques described herein can be used to identify certain tissues, see through tissues in the foreground, estimate tissue geometries and parameters, calculate a three-dimensional topography of a scene, and calculate dimensions and distances for objects within the scene. The advanced visualization techniques described herein specifically include multispectral visualization, fluorescence visualization, laser mapping visualization, and stereo visualization with disparity mapping.

### Multispectral Visualization

Spectral imaging uses multiple bands across the electromagnetic spectrum. This is different from conventional cameras that only capture light across the three wavelength bands based in the visible spectrum that are discernable by the human eye, including the red, green, and blue wavelengths to generate an RGB image. Spectral imaging may use any wavelength bands in the electromagnetic spectrum, including infrared wavelengths, the visible spectrum, the ultraviolet spectrum, x-ray wavelengths, or any suitable combination of various wavelength bands. Spectral imaging may overlay imaging generated based on non-visible bands (e.g., infrared) on top of imaging based on visible bands (e.g., a standard RGB image) to provide additional information that is easily discernable by a person or computer algorithm.

The multispectral imaging techniques discussed herein can be used to “see through” layers of tissue in the foreground of a scene to identify specific types of tissue and/or specific biological or chemical processes. Multispectral imaging can be used in the medical context to quantitatively track the process of a disease and to determine tissue pathology. Additionally, multispectral imaging can be used to identify critical structures such as nerve tissue, muscle tissue, cancerous cells, blood vessels, and so forth. In an embodiment, multispectral partitions of EMR are pulsed and data is gathered regarding the spectral responses of different types of tissue in response to the partitions of EMR. A datastore of spectral responses can be generated and analyzed to assess a scene and predict which tissues are present within the scene based on the sensed spectral responses. Additionally, machine learning, artificial intelligence, and/or deep learning methods may be implemented to process image sensor readings from different wavelengths and then detect a specific tissue class for each pixel. These algorithms are typically trained using expert labeled datasets where tissue spectral signatures are fed to the machine learning algorithm with the corresponding known tissue type.

Multispectral imaging enables numerous advantages over conventional imaging. The information obtained by multispectral imaging enables medical practitioners and/or computer-implemented programs to precisely identify certain tissues or conditions that may not be possible to identify with RGB imaging. Additionally, multispectral imaging may be used during medical procedures to provide image-guided surgery that enables a medical practitioner to, for example, view tissues located behind certain tissues or fluids, identify atypical cancerous cells in contrast with typical healthy cells, identify certain tissues or conditions, identify critical structures, and so forth. Multispectral imaging provides specialized diagnostic information about tissue physiology, morphology, and composition that cannot be generated with conventional imaging.

### Fluorescence Visualization

Fluorescence occurs when an orbital electron of a molecule, atom, or nanostructure is excited by light or other EMR, and then relaxes to its ground state by emitting a photon from the excited state. The specific frequencies of EMR that excite the orbital electron, or are emitted by the photon during relaxation, are dependent on the particular atom, molecule, or nanostructure. In most cases, the light emitted by the substance has a longer wavelength, and therefore lower energy, than the radiation that was absorbed by the substance.

Fluorescence imaging is particularly useful in biochemistry and medicine as a non-destructive means for tracking or analyzing biological molecules. The biological molecules, including certain tissues or structures, are tracked by analyzing the fluorescent emission of the biological molecules after being excited by a certain wavelength of EMR. However, relatively few cellular components are naturally fluorescent. In certain implementations, it may be desirable to visualize a certain tissue, structure, chemical process, or biological process that is not intrinsically fluorescent. In such an implementation, the body may be administered a dye or reagent that may include a molecule, protein, or quantum dot having fluorescent properties. The reagent or dye may then fluoresce after being excited by a certain wavelength of EMR. Different reagents or dyes may include different molecules, proteins, and/or quantum dots that will fluoresce at particular wavelengths of EMR. Thus, it may be necessary to excite the reagent or dye with a specialized band of EMR to achieve fluorescence and identify the desired tissue, structure, or process in the body.

The fluorescence imaging techniques described herein may be used to identify certain materials, tissues, components, or processes within a body cavity or other light deficient environment. Fluorescence imaging data may be provided to a medical practitioner or computer-implemented algorithm to enable the identification of certain structures or tissues within a body. Such fluorescence imaging data may be overlaid on black-and-white or RGB images to provide additional information and context.

The fluorescence imaging techniques described herein may be implemented in coordination with fluorescent reagents or dyes. Some reagents or dyes are known to attach to certain types of tissues and fluoresce at specific wavelengths of the electromagnetic spectrum. In an implementation, a reagent or dye is administered to a patient that is configured to fluoresce when activated by certain wavelengths of light. The visualization system disclosed herein is used to excite and fluoresce the reagent or dye. The fluorescence of the reagent or dye is detected by an image sensor to aid in the identification of tissues or structures in the body cavity. In an implementation, a patient is administered a plurality of reagents or dyes that are each configured to fluoresce at different wavelengths and/or provide an indication of different structures, tissues, chemical reactions, biological processes, and so forth. In such an implementation, the visualization system described herein emits each of the applicable wavelengths to fluoresce each of the applicable reagents or dyes. This may negate the need to perform individual imaging procedures for each of the plurality of reagents or dyes.

### Laser Mapping Visualization

Laser mapping generally includes the controlled deflection of laser beams. Laser mapping can be implemented to generate one or more of a three-dimensional topographical map of a scene, calculate distances between objects within the scene, calculate dimensions of objects within the scene, track the relative locations of tools within the scene, and so forth.

Laser mapping combines controlled steering of laser beams with a laser rangefinder. By taking a distance measurement at every direction, the laser rangefinder can rapidly capture the surface shape of objects, tools, and landscapes. Construction of a full three-dimensional topography may include combining multiple surface models that are obtained from different viewing angles. Various measurement systems and methods exist in the art for applications in archaeology, geography, atmospheric physics, autonomous vehicles, and others. One such system includes light detection and ranging (LIDAR), which is a three-dimensional mapping system. LIDAR has been applied in navigation systems such as airplanes or satellites to determine position and orientation of a sensor in combination with other systems and sensors. LIDAR uses active sensors to illuminate an object and detect energy that is reflected off the object and back to a sensor.

As discussed herein, the term “laser mapping” includes laser tracking. Laser tracking, or the use of lasers for tool tracking, measures objects by determining the positions of optical targets held against those objects. Laser trackers can be accurate to the order of 0.025 mm over a distance of several meters. The visualization system described herein pulses EMR for use in conjunction with a laser tracking system such that the position of tools within a scene can be tracked and measured.

The endoscopic visualization system described herein implements laser mapping imaging to determine precise measurements and topographical outlines of a scene. In one implementation, mapping data is used to determine precise measurements between, for example, structures or organs in a body cavity, devices, or tools in the body cavity, and/or critical structures in the body cavity. As discussed herein, the term “mapping” encompasses technologies referred to as laser mapping, laser scanning, topographical scanning, three-dimensional scanning, laser tracking, tool tracking, and others. A mapping data frame as discussed herein includes data for calculating one or more of a topographical map of a scene, dimensions of objects or structures within a scene, distances between objects or structures within the scene, relative locations of tools or other objects within the scene, and so forth.

Additionally, the systems described herein are capable of calculating a disparity map for generating a three-dimensional rendering of a scene. Disparity is the apparent motion of objects between a pair of stereo images. Given a pair of stereo images, the disparity map is computed by matching each pixel within the “left image” with its corresponding pixel within the “right image.” Then, the distance is computed for each pair of matching pixel. Finally, the disparity map is generated by representing these distance values as an intensity image. The depth is inversely proportional to the disparity, and thus, when the geometric arrangement of the image sensors is known, the disparity map is converted into a depth map using triangulation.

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

Before the structure, systems, and methods are disclosed and described, it is to be understood that this disclosure is not limited to the particular structures, configurations, process steps, and materials disclosed herein as such structures, configurations, process steps, and materials may vary somewhat. It is also to be understood that the terminology employed herein is used for the purpose of describing particular embodiments only and is not intended to be limiting since the scope of the disclosure will be limited only by the appended claims and equivalents thereof.

In describing and claiming the subject matter of the disclosure, the following terminology will be used in accordance with the definitions set out below.

It must be noted that, as used in this specification and the appended claims, the singular forms “a,” “an,” and “the” include plural referents unless the context clearly dictates otherwise.

As used herein, the terms “comprising,” “including,” “containing,” “characterized by,” and grammatical equivalents thereof are inclusive or open-ended terms that do not exclude additional, unrecited elements or method steps.

As used herein, the phrase “consisting of” and grammatical equivalents thereof exclude any element or step not specified in the claim.

As used herein, the phrase “consisting essentially of” and grammatical equivalents thereof limit the scope of a claim to the specified materials or steps and those that do not materially affect the basic and novel characteristic or characteristics of the claimed disclosure.

As used herein, the term “proximal” shall refer broadly to the concept of a portion nearest an origin.

As used herein, the term “distal” shall generally refer to the opposite of proximal, and thus to the concept of a portion farther from an origin, or a farthest portion, depending upon the context.

As used herein, color sensors are sensors known to have a color filter array (CFA) thereon to filter the incoming EMR into its separate components. In the visual range of the electromagnetic spectrum, such a CFA may be built on a Bayer pattern or modification thereon to separate green, red, and blue spectrum components of visible EMR.

As used herein, multispectral sensors are sensors known to have a multispectral filter array (MSFA) comprising a patterned arrangement of optical bandpass filters used to sample multiple selected wavelengths of wavebands of EMR.

As used herein, a monochromatic sensor refers to an unfiltered imaging sensor comprising color-agnostic pixels.

The systems, methods, and devices described herein are specifically optimized to account for variations between “stronger” electromagnetic radiation (EMR) sources and “weaker” EMR sources. In some cases, the stronger EMR sources are considered “stronger” based on the inherent qualities of a pixel array, e.g., if a pixel array is inherently more sensitive to detecting EMR emitted by the stronger EMR source, then the stronger EMR source may be classified as “stronger” when compared with another EMR source. Conversely, if the pixel array is inherently less sensitive to detecting EMR emitted by the weaker EMR source, then the weaker EMR source may be classified as “weaker” when compared with another EMR source. Additionally, a “stronger” EMR source may have a higher amplitude, greater brightness, or higher energy output when compared with a “weaker” EMR source. The present disclosure addresses the disparity between stronger EMR sources and weaker EMR sources by adjusting a pulse cycle of an emitter to ensure a pixel array has sufficient time to accumulate a sufficient amount of EMR corresponding with each of a stronger EMR source and a weaker EMR source.

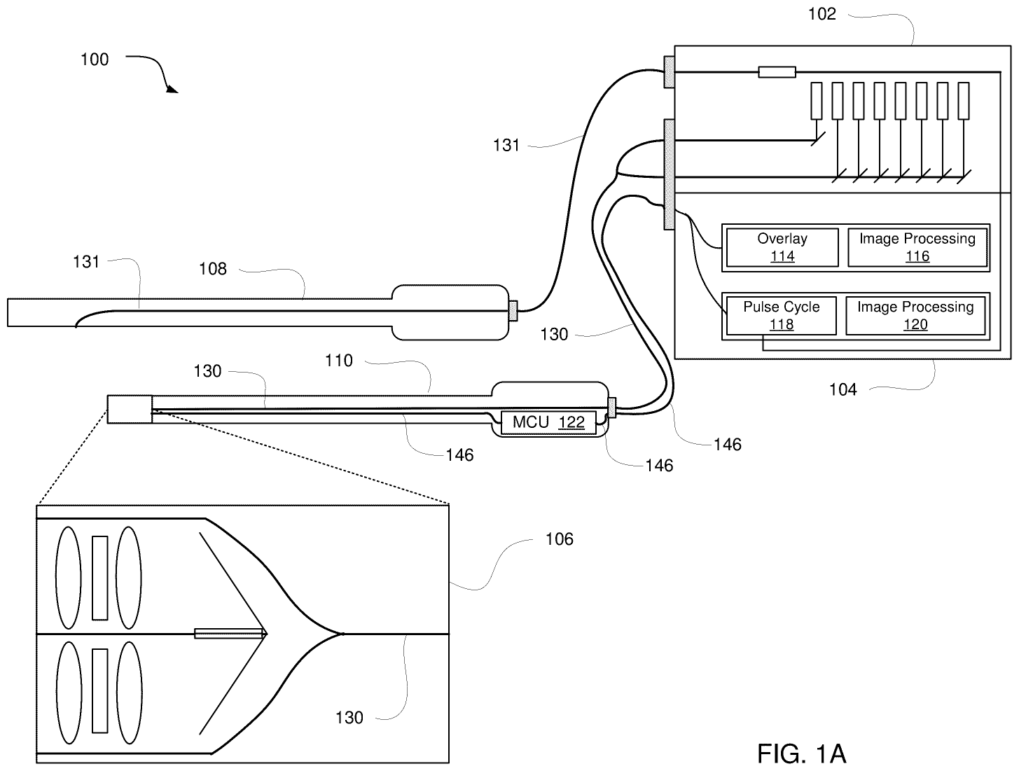

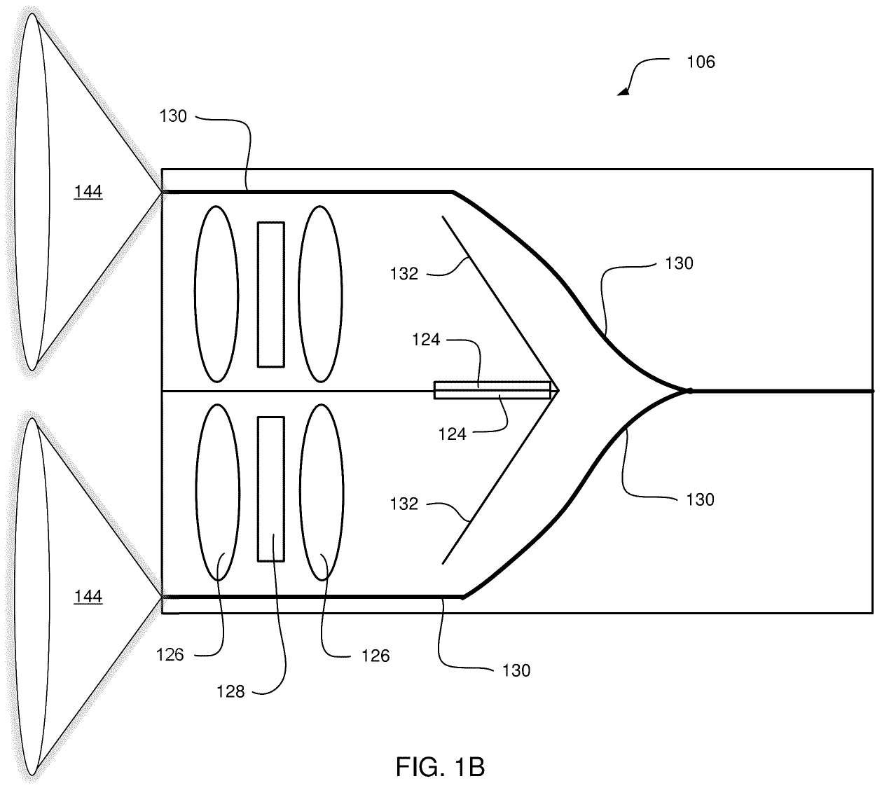

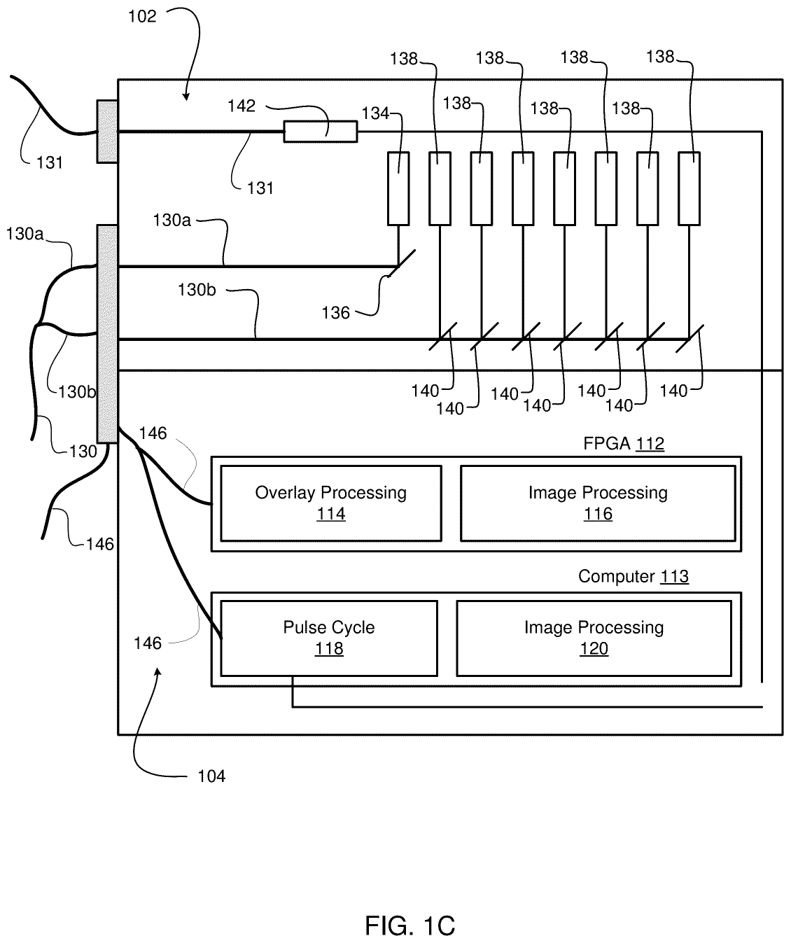

Referring now to the figures, FIGS. 1A-1C illustrate schematic diagrams of a system 100 for endoscopic visualization. The system 100 includes an emitter 102, a controller 104, and an optical visualization system 106. The system 100 includes one or more tools 108, which may include endoscopic tools such as forceps, brushes, scissors, cutters, burs, staplers, ligation devices, tissue staplers, suturing systems, and so forth. The system 100 includes one or more endoscopes 110 such as arthroscopes, bronchoscopes, colonoscopes, colposcopes, cystoscopes, esophagoscope, gastroscopes, laparoscopes, laryngoscopes, neuroendoscopes, proctoscopes, sigmoidoscopes, thoracoscopes, and so forth. The system 100 may include additional endoscopes 110 and/or tools 108 with an image sensor equipped therein. In these implementations, the system 100 is equipped to output stereo visualization data for generating a three-dimensional topographical map of a scene using disparity mapping and triangulation.

The optical visualization system 106 may be disposed at a distal end of a tube of an endoscope 110. Alternatively, one or more components of the optical visualization system 106 may be disposed at a proximal end of the tube of the endoscope 110 or in another region of the endoscope 110. The optical visualization system 106 includes components for directing beams of EMR on to the pixel array 125 of the one or more image sensors 124. The optical visualization system 106 may include any of the lens assembly components described herein.

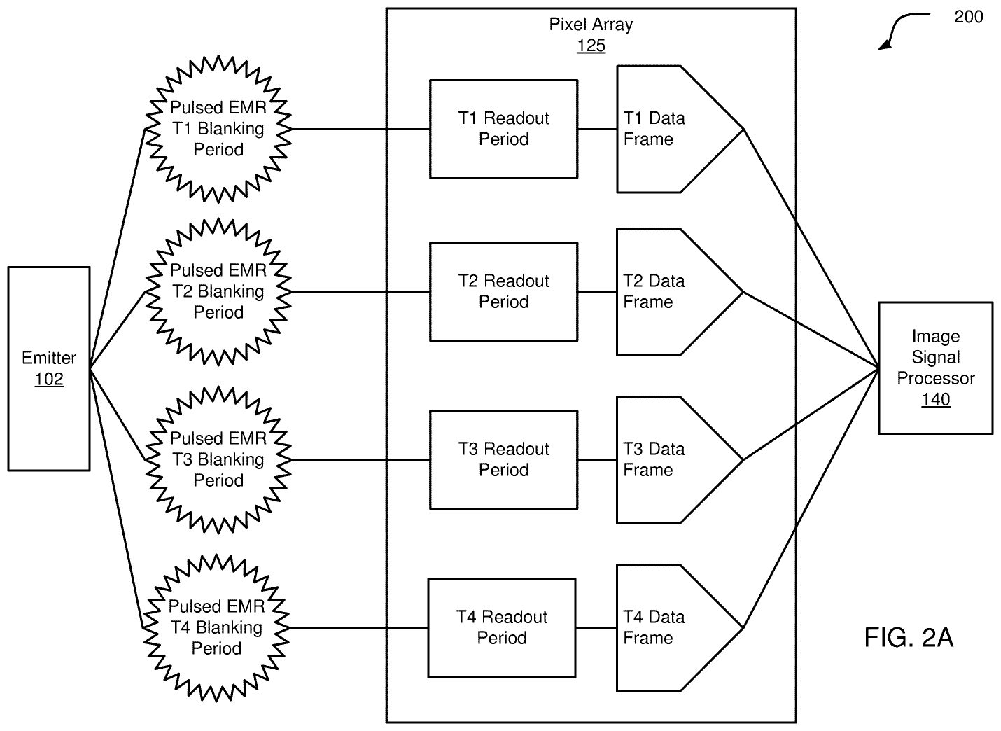

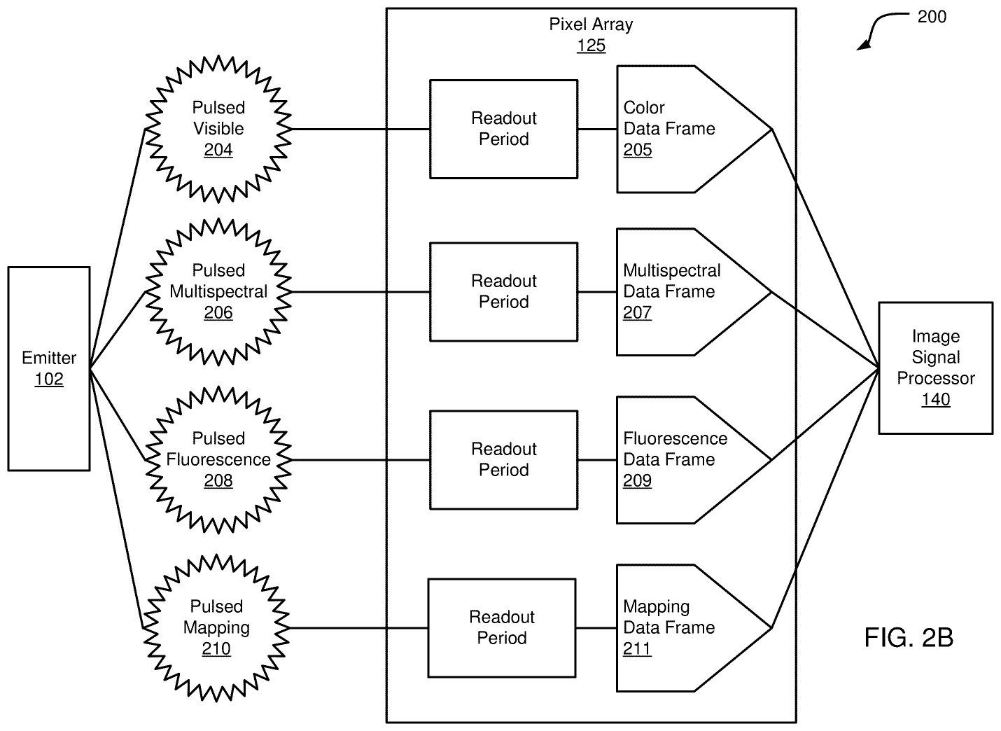

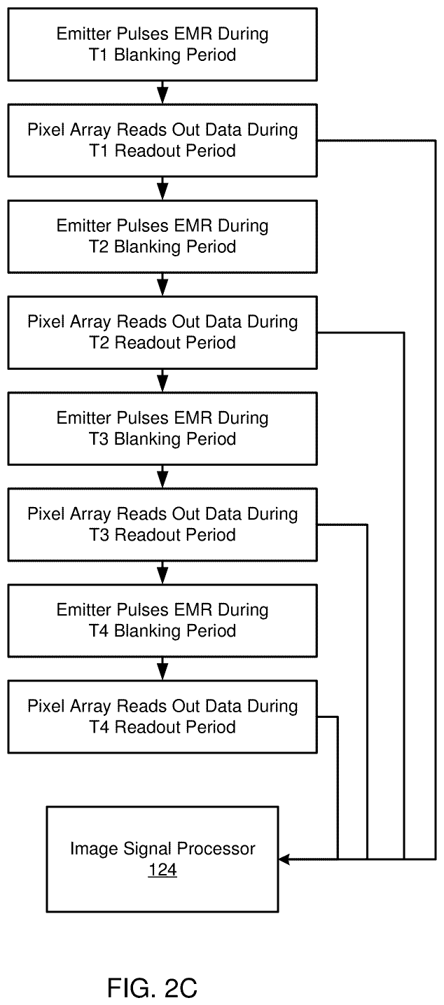

The optical visualization system 106 may include one or more image sensors 124 that each include a pixel array (see pixel array 125 first illustrated in FIG. 2A). The optical visualization system 106 may include one or more lenses 126 and filters 128 and may further include one or more prisms 132 for reflecting EMR on to the pixel array 125 of the one or more image sensors 124. The system 100 may include a waveguide 130 configured to transmit EMR from the emitter 102 to a distal end of the endoscope 110 to illuminate a light deficient environment for visualization, such as within a surgical scene. The system 100 may further include a waveguide 131 configured to transmit EMR from the emitter 102 to a termination point on the tool 108, which may specifically be actuated for laser mapping imaging and tool tracking as described herein.

The optical visualization system 106 may specifically include two lenses 126 dedicated to each image sensor 124 to focus EMR on to a rotated image sensor 124 and enable a depth view. The filter 128 may include a notch filter configured to block unwanted reflected EMR. In a particular use-case, the unwanted reflected EMR may include a fluorescence excitation wavelength that was pulsed by the emitter 102, wherein the system 100 wishes to only detect a fluorescence relaxation wavelength emitted by a fluorescent reagent or tissue.

The optical visualization system 106 may be equipped with a means to exchange the image sensors 124. In some cases, it may be desirable to retrieve one or more of the image sensors 124 and replace it with a different image sensor 124 equipped with a different color filter array (CFA) or multispectral filter array (MSFA). Each image sensor 124 may be equipped with a different MSFA that is configured to identify a certain tissue, biological process, reagent, chemical process, or condition based on spectral response signatures. In some cases, it may be desirable to utilize different image sensors 124 equipped with different MSFAs. In some cases, one or more of the image sensors 124 is equipped with tunable filters that may be adjusted in real-time to transmit different wavelengths of EMR to the pixel array 125.

The optical visualization system 106 may additionally include an inertial measurement unit (IMU) (not shown). The IMU may be configured to track the real-time movements and rotations of the image sensor 124. Sensor data output from the IMU may be provided to the controller 104 to improve post processing of image frames output by the image sensor 124. Specifically, sensor data captured by the IMU may be utilized to stabilize the movement of image frames and/or the movement of false color overlays rendered over color image frames.

The image sensor 124 includes one or more image sensors, and the example implementation illustrated in FIGS. 1A-1B illustrates an optical visualization system 106 comprising two image sensors 124. The image sensor 124 may include a CMOS image sensor and may specifically include a high-resolution image sensor configured to read out data according to a rolling readout scheme. The image sensors 124 may include a plurality of different image sensors that are tuned to collect different wavebands of EMR with varying efficiencies. In an implementation, the image sensors 124 include separate image sensors that are optimized for color imaging, fluorescence imaging, multispectral imaging, and/or topographical mapping.

The optical visualization system 106 typically includes multiple image sensors 124 such that the system 100 is equipped to output stereo visualization data. In some cases, stereo data frames are assessed to output a disparity map showing apparent motion of objects between the “left” stereo image and the “right” stereo image. Because the geographical locations of the image sensors 124 is known, the disparity map may then be used to generate a three-dimensional topographical map of a scene using triangulation.

The emitter 102 includes one or more EMR sources, which may include, for example, lasers, laser bundles, light emitting diodes (LEDs), electric discharge sources, incandescence sources, electroluminescence sources, and so forth. In some implementations, the emitter 102 includes at least one white EMR source 134 (may be referred to herein as a white light source). The emitter 102 may additionally include one or more EMR sources 138 that are tuned to emit a certain waveband of EMR. The EMR sources 138 may specifically be tuned to emit a waveband of EMR that is selected for multispectral or fluorescence visualization. The emitter 102 may additionally include one or more mapping sources 142 that are configured to emit EMR in a mapping pattern such as a grid array or dot array selected for capturing data for topographical mapping or anatomical measurement.

The EMR sources 138 that are tuned to emit a waveband of EMR that is selected for fluorescence visualization will be configured to emit only EMR within a fluorescence excitation waveband. The fluorescence excitation waveband is determined based on the known excitation waveband for a certain reagent or tissue.

The EMR sources 138 that are tuned to emit a waveband of EMR that is selected for multispectral visualization will be configured to emit only EMR within a waveband that corresponds with the spectral reflectance waveband of a tissue, chemical process, or biological process. The spectral reflectance waveband may be referred to as the “spectral reflectance signature” of the tissue, chemical process, or biological process. The spectral reflectance signature for the tissue, chemical process, or biological process is determined based on one or more of: the ratio of upwelling to down-welling radiant fluxes for the given tissue, chemical process, or biological process; the illumination and observation properties for the given tissue, chemical process, or biological process; or the spectral sampling properties of the image sensor 124.

Some components of a scene (e.g., tissues, chemical processes, or biological processes) are known to reflect EMR within an identified narrow waveband. The system 100 identifies these components by causing the emitter 102 to pulse EMR within the identified narrow waveband and then causing the image sensor 124 to accumulate the spectral reflectance of the component. The pixel integration data output by the image sensor 124 will indicate which pixels accumulated the spectral reflectance for the component, and thus, the system 100 is capable of identifying the component within the scene and/or determining where the component is located within the scene. The spectral reflectance waveband of some components (i.e., the wavelengths of EMR the component is known to reflect) may be very narrow, and in some cases may be 40 nm wide or less, 30 nm wide or less, 20 nm wide or less, 10 nm wide or less, or 5 nm wide or less. Thus, the EMR sources 138 may include narrowband sources tuned to emit only EMR within the narrow spectral reflectance waveband of the component to be identified.

The one or more white EMR sources 134 emit EMR into a dichroic mirror 136 that feeds the white EMR into a waveguide 130, which may specifically include a fiber optic cable or other means for carrying EMR to the endoscope. The white EMR source 134 may specifically feed into a first waveguide 130a dedicated to white EMR. The EMR sources 138 emit EMR into independent dichroic mirrors 140 that each feed EMR into the waveguide 130 and may specifically feed into a second waveguide 130b. The first waveguide 130a and the second waveguide 130b later merge into a waveguide 130 that transmits EMR to a distal end of the endoscope 110 to illuminate a scene with an emission of EMR 144.

The one or more EMR sources 138 that are tuned to emit a waveband of EMR may specifically be tuned to emit EMR that is selected for multispectral or fluorescence visualization. In some cases, the EMR sources 138 are finely tuned to emit a central wavelength of EMR with a tolerance threshold not exceeding ±5 nm, ±4 nm, ±3 nm, ±2 nm, or ±1 nm. The EMR sources 138 may include lasers or laser bundles that are separately cycled on and off by the emitter 102 to pulse the emission of EMR 144 and illuminate a scene with a finely tuned waveband of EMR.

The one or more mapping sources 142 are configured to pulse EMR in a mapping pattern, which may include a dot array, grid array, vertical hashing, horizontal hashing, pin grid array, and so forth. The mapping pattern is selected for laser mapping imaging to determine one or more of a three-dimensional topographical map of a scene, a distance between two or more objects within a scene, a dimension of an object within a scene, a location of a tool 108 within the scene, and so forth. The EMR pulsed by the mapping source 142 is diffracted to spread the energy waves according to the desired mapping pattern. The mapping source 142 may specifically include a device that splits the EMR beam with quantum-dot-array diffraction grafting. The mapping source 142 may be configured to emit low mode laser light.

The controller 104 (may be referred to herein as a camera control unit or CCU) may include a field programmable gate array (FGPA) 112 and a computer 113. The FGPA 112 may be configured to perform overlay processing 114 and image processing 116. The computer 113 may be configured to generate a pulse cycle 118 for the emitter 102 and to perform further image processing 120. The FGPA 112 receives data from the image sensor 124 and may combine data from two or more data frames by way of overlay processing 114 to output an overlay image frame. The computer 113 may provide data to the emitter 102 and the image sensor 124. Specifically, the computer 113 may calculate and adjust a variable pulse cycle to be emitted by the emitter 102 in real-time based on user input. Additionally, the computer 113 may receive data frames from the image sensor 124 and perform further image processing 120 on those data frames.

The controller 104 may communicate with a microcontroller unit (MCU) 122 disposed within a handpiece of the endoscope and/or the image sensor 124 by way of a data transmission pipeline 146. The data transmission pipeline 146 may include a data connection port disposed within a housing of the emitter 102 or the controller 104 that enables a corresponding data cable to carry data to the endoscope 110. In another embodiment, the controller 104 wirelessly communicates with the MCU 122 and/or the image sensor 124 to provide instructions for upcoming data frames. One frame period includes a blanking period and a readout period. Generally speaking, the pixel array 125 accumulates EMR during the blanking period and reads out pixel data during the readout period. It will be understood that a blanking period corresponds to a time between a readout of a last row of active pixels in the pixel array of the image sensor and a beginning of a next subsequent readout of active pixels in the pixel array. Additionally, the readout period corresponds to a duration of time when active pixels in the pixel array are being read. Further, the controller 104 may write correct registers to the image sensor 124 to adjust the duration of one or more of the blanking period or the readout period for each frame period on a frame-by-frame basis within the sensor cycle as needed.

The controller 104 may be in communication with a network, such as the Internet, and automatically upload data to the network for remote storage. The MCU 122 and image sensors 124 may be exchanged and updated and continue to communicate with an established controller 104. In some cases, the controller 104 is “out of date” with respect to the MCU 122 but will still successfully communicate with the MCU 122. This may increase the data security for a hospital or other healthcare facility because the existing controller 104 may be configured to undergo extensive security protocols to protect patient data.

The controller 104 may reprogram the image sensor 124 for each data frame to set a required blanking period duration and/or readout period duration for a subsequent frame period. In some cases, the controller 104 reprograms the image sensor 124 by first sending information to the MCU 122, and then the MCU 122 communicates directly with the image sensor 124 to rewrite registers on the image sensor 124 for an upcoming data frame.

The MCU 122 may be disposed within a handpiece portion of the endoscope 110 and communicate with electronic circuitry (such as the image sensor 124) disposed within a distal end of a tube of the endoscope 110. The MCU 122 receives instructions from the controller 104, including an indication of the pulse cycle 118 provided to the emitter 102 and the corresponding sensor cycle timing for the image sensor 124. The MCU 122 executes a common Application Program Interface (API). The controller 104 communicates with the MCU 122, and the MCU 122 executes a translation function that translates instructions received from the controller 104 into the correct format for each type of image sensor 124. In some cases, the system 100 may include multiple different image sensors that each operate according to a different “language” or formatting, and the MCU 122 is configured to translate instructions from the controller 104 into each of the appropriate data formatting languages. The common API on the MCU 122 passes information by the scene, including, for example parameters pertaining to gain, exposure, white balance, setpoint, and so forth. The MCU 122 runs a feedback algorithm to the controller 104 for any number of parameters depending on the type of visualization.

The MCU 122 stores operational data and images captured by the image sensors 124. In some cases, the MCU 122 does not need to continuously push data up the data chain to the controller 104. The data may be set once on the microcontroller 122, and then only critical information may be pushed through a feedback loop to the controller 104. The MCU 122 may be set up in multiple modes, including a primary mode (may be referred to as a “master” mode when referring to a master/slave communication protocol). The MCU 122 ensures that all downstream components (i.e., distal components including the image sensors 124, which may be referred to as “slaves” in the master/slave communication protocol) are apprised of the configurations for upcoming data frames. The upcoming configurations may include, for example, gain, exposure duration, readout duration, pixel binning configuration, and so forth.

The MCU 122 includes internal logic for executing triggers to coordinate different devices, including, for example multiple image sensors 124. The MCU 122 provides instructions for upcoming frames and executes triggers to ensure that each image sensor 124 begins to capture data the same time. In some cases, the image sensors 124 may automatically advance to a subsequent data frame without receiving a unique trigger from the MCU 122.

In some cases, the endoscope 110 includes two or more image sensors 124 that detect EMR and output data frames simultaneously. The simultaneous data frames may be used to output a three-dimensional image and/or output imagery with increased definition and dynamic range. The pixel array of the image sensor 124 may include active pixels and optical black (“OB”) or optically blind pixels. The optical black pixels may be read during a blanking period of the pixel array when the pixel array is “reset” or calibrated. After the optical black pixels have been read, the active pixels are read during a readout period of the pixel array. The active pixels accumulate EMR that is pulsed by the emitter 102 during the blanking period of the image sensor 124. The pixel array 125 may include monochromatic or “color agnostic” pixels that do not comprise any filter for selectively receiving certain wavebands of EMR. The pixel array may include a color filter array (CFA), such as a Bayer pattern CFA, that selectively allows certain wavebands of EMR to pass through the filters and be accumulated by the pixel array. The pixel array may include a multispectral filter array (MSFA) comprising optical bandpass filters used to sample multiple spectral wavelengths or spectral wavebands of EMR.

The image sensor 124 is instructed by a combination of the MCU 122 and the controller 104 working in a coordinated effort. Ultimately, the MCU 122 provides the image sensor 124 with instructions on how to capture the upcoming data frame. These instructions include, for example, an indication of the gain, exposure, white balance, exposure duration, readout duration, pixel binning configuration, and so forth for the upcoming data frame. When the image sensor 124 is reading out data for a current data frame, the MCU 122 is rewriting the correct registers for the next data frame. The MCU 122 and the image sensor 124 operate in a back-and-forth data flow, wherein the image sensor 124 provides data to the MCU 122 and the MCU 122 rewrites correct registers to the image sensor 124 for each upcoming data frame. The MCU 122 and the image sensor 124 may operate according to a “ping pong buffer” in some configurations.

The image sensor 124, MCU 122, and controller 104 engage in a feedback loop to continuously adjust and optimize configurations for upcoming data frames based on output data. The MCU 122 continually rewrites correct registers to the image sensor 124 depending on the type of upcoming data frame (i.e., color data frame, multispectral data frame, fluorescence data frame, topographical mapping data frame, and so forth), configurations for previously output data frames, and user input. In an example implementation, the image sensor 124 outputs a multispectral data frame in response to the emitter 102 pulsing a multispectral waveband of EMR. The MCU 122 and/or controller 104 determines that the multispectral data frame is underexposed and cannot successfully be analyzed by a corresponding machine learning algorithm. The MCU 122 and/or controller 104 then adjusts configurations for upcoming multispectral data frames to ensure that future multispectral data frames are properly exposed. The MCU 122 and/or controller 104 may indicate that the gain, exposure duration, pixel binning configuration, etc. must be adjusted for future multispectral data frames to ensure proper exposure. All image sensor 124 configurations may be adjusted in real-time based on previously output data processed through the feedback loop, and further based on user input.

The waveguides 130, 131 include one or more optical fibers. The optical fibers may be made of a low-cost material, such as plastic to allow for disposal of one or more of the waveguides 130, 131. In some implementations, one or more of the waveguides 130, 131 include a single glass fiber having a diameter of 500 microns. In some implementations, one or more of the waveguides 130, 131 include a plurality of glass fibers.

FIGS. 2A and 2B each illustrate a schematic diagram of a data flow 200 for time-sequenced visualization of a light deficient environment. The data flow 200 illustrated in FIGS. 2A-2B may be implemented by the system 100 for endoscopic visualization illustrated in FIGS. 1A-1C. FIG. 2A illustrates a generic implementation that may be applied to any type of illumination or wavelengths of EMR. FIG. 2B illustrates an example implementation wherein the emitter 102 actuates visible, multispectral, fluorescence, and mapping EMR sources.

The data flow 200 includes an emitter 102, a pixel array 125 of an image sensor 124 (not shown), and an image signal processor 140. The image signal processor 140 may include one or more of the image processing 116, 120 modules illustrated in FIGS. 1A and 1C. The emitter 102 includes a plurality of separate and independently actuatable EMR sources (see, e.g., 134, 138 illustrated in FIGS. 1A and 1C). Each of the EMR sources can be cycled on and off to emit a pulse of EMR with a defined duration and magnitude. The pixel array 125 of the image sensor 124 may include a color filter array (CFA) or an unfiltered array comprising color-agnostic pixels. The emitter 102 and the pixel array 125 are each in communication with a controller 104 (not shown in FIGS. 2A-2B) that instructs the emitter 102 and the pixel array 125 to synchronize operations to generate a plurality of data frames according to a desired visualization scheme.

The controller 104 instructs the emitter 102 to cycle the plurality of EMR sources according to a variable pulse cycle. The controller 104 calculates the variable pulse cycle based at least in part upon a user input indicating the desired visualization scheme. For example, the desired visualization scheme may indicate the user wishes to view a scene with only color imaging. In this case, the variable pulse cycle may include only pulses of white EMR. In an alternative example, the desired visualization scheme may indicate the user wishes to be notified when nerve tissue can be identified in the scene and/or when a tool within the scene is within a threshold distance from the nerve tissue. In this example, the variable pulse cycle may include pulses of white EMR and may further include pulses of one or more multispectral wavebands of EMR that elicit a spectral response from the nerve tissue and/or “see through” non-nerve tissues by penetrating those non-nerve tissues. Additionally, the variable pulse cycle may include pulses of EMR in a mapping pattern configured for laser mapping imaging to determine when the tool is within the threshold distance from the nerve tissue. The controller 104 may reconfigure the variable pulse cycle in real-time in response to receiving a revised desired visualization scheme from the user.

FIG. 2A illustrates wherein the emitter cycles one or more EMR sources on and off to emit a pulse of EMR during each of a plurality of separate blanking periods of the pixel array 125. Specifically, the emitter 102 emits pulsed EMR during each of a T1 blanking period, T2 blanking period, T3 blanking period, and T4 blanking period of the pixel array 125. The pixel array 125 accumulates EMR during its blanking periods and reads out data during its readout periods.

Specifically, the pixel array 125 accumulates EMR during the T1 blanking period and reads out the T1 data frame during the T1 readout period, which follows the T1 blanking period. Similarly, the pixel array 125 accumulates EMR during the T2 blanking period and reads out the T2 data frame during the T2 readout period, which follows the T2 blanking period. The pixel array 125 accumulates EMR during the T3 blanking period and reads out the T3 data frame during the T3 readout period, which follows the T3 blanking period. The pixel array 125 accumulates EMR during the T4 blanking period and reads out the T4 data frame during the T4 readout period, which follows the T4 blanking period. Each of the T1 data frame, the T2 data frame, the T3 data frame, and the T4 data frame is provided to the image signal processor 140.

The contents of each of the T1-T4 data frames is dependent on the type of EMR that was pulsed by the emitter 102 during the preceding blanking period. For example, if the emitter 102 pulses white light during the preceding blanking period, then the resultant data frame may include a color data frame (if the pixel array 125 includes a color filter array for outputting red, green, and blue image data). Further for example, if the emitter 102 pulses a multispectral waveband of EMR during the preceding blanking period, then the resultant data frame is a multispectral data frame comprising information for identifying a spectral response by one or more objects within the scene and/or information for “seeing through” one or more structures within the scene. Further for example, if the emitter 102 pulses a fluorescence excitation waveband of EMR during the preceding blanking period, then the resultant data frame is a fluorescence data frame comprising information for identifying a fluorescent reagent or autofluorescence response by a tissue within the scene. Further for example, if the emitter 102 pulses EMR in a mapping pattern during the preceding blanking period, then the resultant data frame is a mapping data frame comprising information for calculating one or more of a three-dimensional topographical map of the scene, a dimension of one or more objects within the scene, a distance between two or more objects within the scene, and so forth.

Some “machine vision” or “computer vision” data frames, including multispectral data frames, fluorescence data frames, and mapping data frames may be provided to a corresponding algorithm or neural network configured to evaluate the information therein. A multispectral algorithm may be configured to identify one or more tissue structures within a scene based on how those tissue structures respond to one or more different wavebands of EMR selected for multispectral imaging. A fluorescence algorithm may be configured to identify a location of a fluorescent reagent or auto-fluorescing tissue structure within a scene. A mapping algorithm may be configured to calculate one or more of a three-dimensional topographical map of a scene, a depth map, a dimension of one or more objects within the scene, and/or a distance between two or more objects within the scene based on the mapping data frame.

FIG. 2B illustrates an example wherein the emitter 102 cycles separate visible, multispectral, fluorescence, and mapping EMR sources to emit pulsed visible 204, pulsed multispectral 206, pulsed fluorescence 208, and pulsed EMR in a mapping pattern 210. It should be appreciated that FIG. 2B is illustrative only, and that the emissions 204, 206, 208, 210 may be emitted in any order, may be emitted during a single visualization session as shown in FIG. 2B, and may be emitted during separate visualization sessions.

The pixel array 125 reads out a color data frame 205 in response to the emitter 102 pulsing the pulsed visible 204 EMR. The pulsed visible 204 EMR may specifically include a pulse of white light. The pixel array 125 reads out a multispectral data frame 207 in response to the emitter 102 pulsing the multispectral 206 waveband of EMR. The pulsed multispectral 206 waveband of EMR may specifically include one or more of EMR within a waveband from about 513-545 nanometers (nm), 565-585 nm, 770-790 nm, and/or 900-1000 nm. It will be appreciated that the pulsed multispectral 206 waveband of EMR may include various other wavebands used to elicit a spectral response. The pixel array 125 reads out a fluorescence data frame 209 in response to the emitter 102 pulsing the fluorescence 208 waveband of EMR. The pulsed fluorescence 208 waveband of EMR may specifically include one or more of EMR within a waveband from about 770-795 nm and/or 790-815 nm. The pixel array 125 reads out a mapping data frame 211 in response to the emitter 102 pulsing EMR in a mapping pattern 210. The pulsed mapping pattern 210 may include one or more of vertical hashing, horizontal hashing, a pin grid array, a dot array, a raster grid of discrete points, and so forth. Each of the color data frame 205, the multispectral data frame 207, the fluorescence data frame 209, and the mapping data frame 211 is provided to the image signal processor 140.

In an implementation, the emitter 102 separately pulses red, green, and blue visible EMR. In this implementation, the pixel array 125 may include a monochromatic (color agnostic) array of pixels. The pixel array 125 may separately read out a red data frame, a green data frame, and a blue data frame in response to the separate pulses of red, green, and blue visible EMR.

In an implementation, the emitter 102 separately pulses wavebands of visible EMR that are selected for capturing luminance (“Y”) imaging data, red chrominance (“Cr”) imaging data, and blue chrominance (“Cb”) imaging data. In this implementation, the pixel array 125 may separately read out a luminance data frame (comprising only luminance imaging information), a red chrominance data frame, and a blue chrominance data frame.

FIG. 2C illustrates a schematic flow chart diagram of a process flow for synchronizing operations of the emitter 102 and the pixel array 125. The process flow corresponds with the schematic diagram illustrated in FIG. 2A. The process flow includes the controller 104 instructing the emitter 102 to pulse EMR during a T1 blanking period of the pixel array 125 and then instructing the pixel array 125 to read out data during a T1 readout period following the T1 blanking period. Similarly, the controller 104 instructs the emitter to pulse EMR during each of the T2 blanking period, the T3 blanking period, and the T4 blanking period. The controller 104 instructs the emitter to read out data during each of the T2 readout period, the T3 readout period, and the T4 readout period that follow the corresponding blanking periods. Each of the output data frames are provided to the image signal processor 140.

The emitter 102 pulses according to a variable pulse cycle that includes one or more types of EMR. The variable pulse cycle may include visible EMR, which may include a white light emission, red light emission, green light emission, blue light emission, or some other waveband of visible EMR. The white light emission may be pulsed with a white light emitting diode (LED) or other light source and may alternatively be pulsed with a combination of red, green, and blue light sources pulsing in concert. The variable pulse cycle may include one or more wavebands of EMR that are selected for multispectral imaging or fluorescence imaging. The variable pulse cycle may include one or more emissions of EMR in a mapping pattern selected for three-dimensional topographical mapping or calculating dimensions within a scene. In some cases, several types of EMR are represented in the variable pulse cycle with different regularity than other types of EMR. This may be implemented to emphasize and de-emphasize aspects of the recorded scene as desired by the user.

The controller 104 adjusts the variable pulse cycle in real-time based on the visualization objectives. The system enables a user to input one or more visualization objectives and to change those objectives while using the system. For example, the visualization objective may indicate the user wishes to view only color imaging data, and in this case, the variable pulse cycle may include pulsed or constant emissions of white light (or other visible EMR). The visualization objective may indicate the user wishes to be notified when a scene includes one or more types of tissue or conditions that may be identified using one or more of color imaging, multispectral imaging, or fluorescence imaging. The visualization objective may indicate that a patient has been administered a certain fluorescent reagent or dye, and that fluorescence imaging should continue while the reagent or dye remains active. The visualization objective may indicate the user wishes to view a three-dimensional topographical map of a scene, receive information regarding distances or dimensions within the scene, receive an alert when a tool comes within critical distance from a certain tissue structure, and so forth.

The variable pulse cycle may include one or more finely tuned partitions of the electromagnetic spectrum that are selected to elicit a fluorescence response from a reagent, dye, or auto-fluorescing tissue. The fluorescence excitation wavebands of EMR include one or more of the following: 400±50 nm, 450±50 nm, 500±50 nm, 550±50 nm, 600±50 nm, 650±50 nm, 700±50 nm, 710±50 nm, 720±50 nm, 730±50 nm, 740±50 nm, 750±50 nm, 760±50 nm, 770±50 nm, 780±50 nm, 790±50 nm, 800±50 nm, 810±50 nm, 820±50 nm, 830±50 nm, 840±50 nm, 850±50 nm, 860±50 nm, 870±50 nm, 880±50 nm, 890±50 nm, 900±50 nm, 910±50, 920±50, 930±50, 940±50, 950±50, 960±50, 970±50, 980±50, 990±50, or 1000±50. The aforementioned wavebands may be finely tuned such that the emitter pulses the central wavelength with a tolerance threshold of ±100 nm, ±90 nm, ±80 nm, ±70 nm, ±60 nm, ±50 nm, ±40 nm, ±30 nm, ±20 nm, ±10 nm, ±8 nm, ±6 nm, ±5 nm, ±4 nm, ±3 nm, ±2 nm, ±1 nm, and so forth. In some cases, the emitter includes a plurality of laser bundles that are each configured to pulse a particular wavelength of EMR with a tolerance threshold not greater than ±5 nm, ±4 nm, ±3 nm, or ±2 nm.

The variable pulse cycle may include one or more wavebands of EMR that are tuned for multispectral imaging. These wavebands of EMR are selected to elicit a spectral response from a certain tissue or penetrate through a certain tissue (such that substances disposed behind that tissue may be visualized). The multispectral wavebands of EMR include one or more of the following: 400±50 nm, 410±50 nm, 420±50 nm, 430±50 nm, 440±50 nm, 450±50 nm, 460±50 nm, 470±50 nm, 480±50 nm, 490±50 nm, 500±50 nm, 510±50 nm, 520±50 nm, 530±50 nm, 540±50 nm, 550±50 nm, 560±50 nm, 570±50 nm, 580±50 nm, 590±50 nm, 600±50 nm, 610±50 nm, 620±50 nm, 630±50 nm, 640±50 nm, 650±50 nm, 660±50 nm, 670±50 nm, 680±50 nm, 690±50 nm, 700±50 nm, 710±50 nm, 720±50 nm, 730±50 nm, 740±50 nm, 750±50 nm, 760±50 nm, 770±50 nm, 780±50 nm, 790±50 nm, 800±50 nm, 810±50 nm, 820±50 nm, 830±50 nm, 840±50 nm, 850±50 nm, 860±50 nm, 870±50 nm, 880±50 nm, 890±50 nm, 900±50 nm, 910±50 nm, 920±50 nm, 930±50 nm, 940±50 nm, 950±50 nm, 960±50 nm, 970±50 nm, 980±50 nm, 990±50 nm, 1000±50 nm, 900±100 nm, 950±100 nm, or 1000±100 nm. The aforementioned wavebands may be finely tuned such that the emitter pulses the central wavelength with a tolerance threshold of ±100 nm, ±90 nm, ±80 nm, ±70 nm, ±60 nm, ±50 nm, ±40 nm, ±30 nm, ±20 nm, ±10 nm, ±8 nm, ±6 nm, ±5 nm, ±4 nm, ±3 nm, ±2 nm, ±1 nm, and so forth. In some cases, the emitter includes a plurality of laser bundles that are each configured to pulse a particular wavelength of EMR with a tolerance threshold not greater than ±5 nm, ±4 nm, ±3 nm, or ±2 nm.

Certain multispectral wavelengths pierce through tissue and enable a medical practitioner to “see through” tissues in the foreground to identify chemical processes, structures, compounds, biological processes, and so forth that are located behind the foreground tissues. The multispectral wavelengths may be specifically selected to identify a specific disease, tissue condition, biological process, chemical process, type of tissue, and so forth that is known to have a certain spectral response.

The variable pulse cycle may include one or more emissions of EMR that are optimized for mapping imaging, which includes, for example, three-dimensional topographical mapping, depth map generation, calculating distances between objects within a scene, calculating dimensions of objects within a scene, determining whether a tool or other object approaches a threshold distance from another object, and so forth. The pulses for laser mapping imaging include EMR formed in a mapping pattern, which may include one or more of vertical hashing, horizontal hashing, a dot array, and so forth.

The controller 104 optimizes the variable pulse cycle to accommodate various imaging and video standards. In most use-cases, the system outputs a video stream comprising at least 30 frames per second (fps). The controller 104 synchronizes operations of the emitter and the image sensor to output data at a sufficient frame rate for visualizing the scene and further for processing the scene with one or more advanced visualization techniques. A user may request a real-time color video stream of the scene and may further request information based on one or more of multispectral imaging, fluorescence imaging, or laser mapping imaging (which may include topographical mapping, calculating dimensions and distances, and so forth). The controller 104 causes the image sensor to separately sense color data frames, multispectral data frames, fluorescence data frames, and mapping data frames based on the variable pulse cycle of the emitter.

In some cases, a user requests more data types than the system can accommodate while maintaining a smooth video frame rate. The system is constrained by the image sensor's ability to accumulate a sufficient amount of electromagnetic energy during each blanking period to output a data frame with sufficient exposure. In some cases, the image sensor outputs data at a rate of 60-120 fps and may specifically output data at a rate of 60 fps. In these cases, for example, the controller 104 may devote 24-30 fps to color visualization and may devote the other frames per second to one or more advanced visualization techniques.

The controller 104 calculates and adjusts the variable pulse cycle of the emitter 102 in real-time based at least in part on the known capabilities of the pixel array 125. The controller 104 may access data stored in memory indicating how long the pixel array 125 must be exposed to a certain waveband of EMR for the pixel array 125 to accumulate a sufficient amount of EMR to output a data frame with sufficient exposure. In most cases, the pixel array 125 is inherently more or less sensitive to different wavebands of EMR. Thus, the pixel array 125 may require a longer or shorter blanking period duration for some wavebands of EMR to ensure that all data frames output by the image sensor 124 comprise sufficient exposure levels.

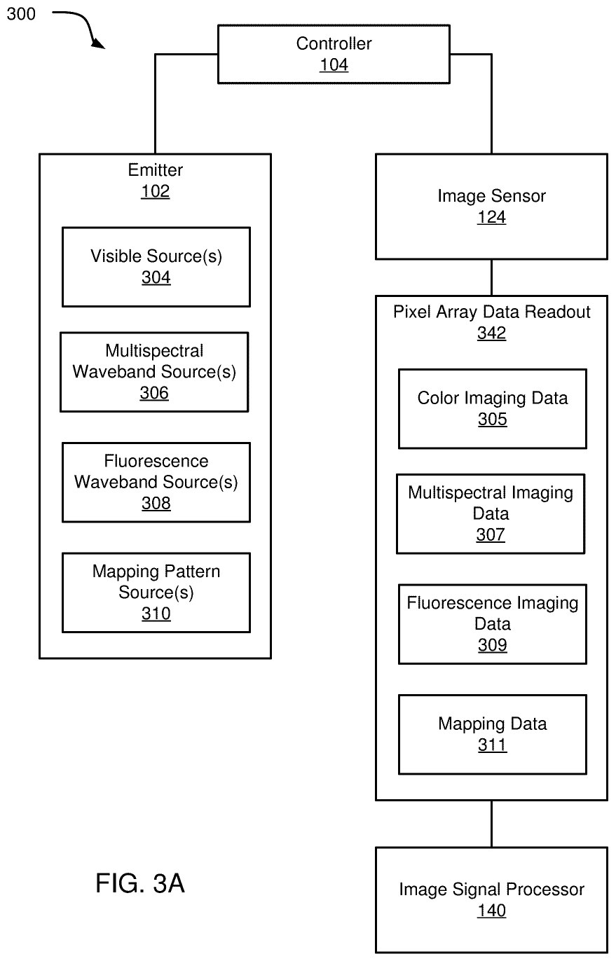

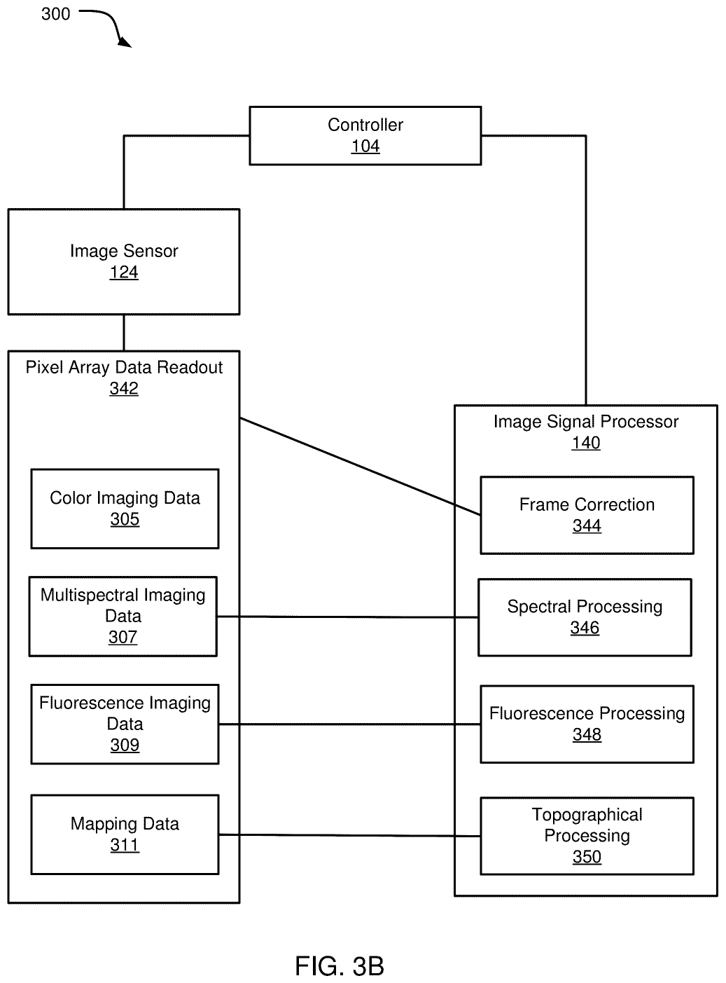

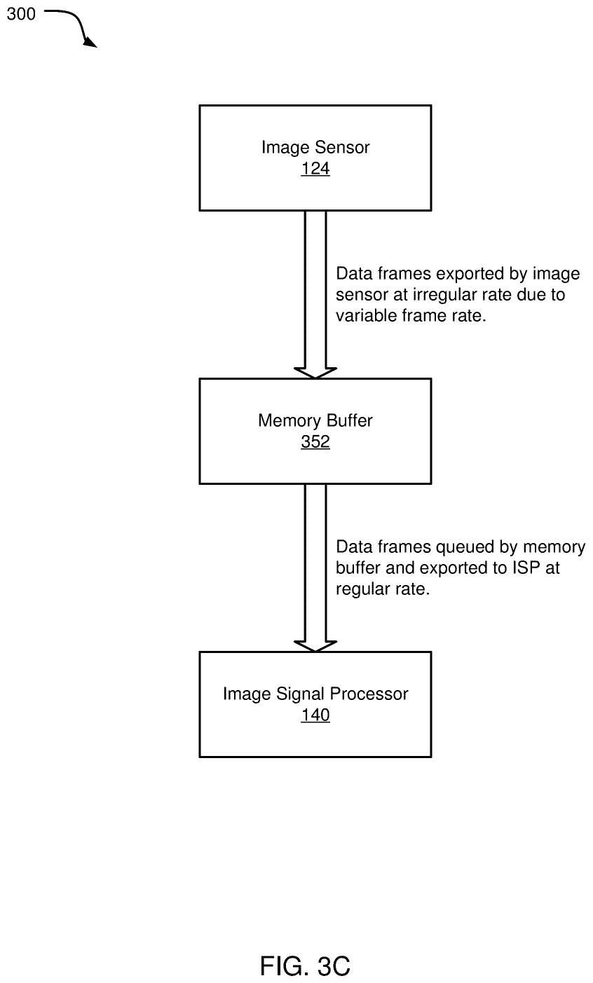

The controller 104 determines the data input requirements for various advanced visualization algorithms (see, e.g., the algorithms 346, 348, 350 first described in FIG. 3B). For example, the controller 104 may determine that certain advanced visualization algorithms do not require a data input at the same regularity as a color video stream output of 30 fps. In these cases, the controller 104 may optimize the variable pulse cycle to include white light pulses at a more frequent rate than pulses for advanced visualization such as multispectral, fluorescence, or laser mapping imaging. Additionally, the controller 104 determines whether certain algorithms may operate with lower resolution data frames that are read out by the image sensor using a pixel binning configuration. In some cases, the controller 104 ensures that all color frames provided to a user are read out in high-resolution (without pixel binning). However, some advanced visualization algorithms (see e.g., 346, 348, 350) may execute with lower resolution data frames.

The system 100 may include a plurality of image sensors 124 that may have different or identical pixel array configurations. For example, one image sensor 124 may include a monochromatic or “color agnostic” pixel array with no filters, another image sensor 124 may include a pixel array with a Bayer pattern CFA, and another image sensor 124 may include a pixel array with a different CFA. The multiple image sensors 124 may be assigned to detect EMR for a certain imaging modality, such as color imaging, multispectral imaging, fluorescence imaging, or laser mapping imaging. Further, each of the image sensors 124 may be configured to simultaneously accumulate EMR and output a data frame, such that all image sensors are capable of sensing data for all imaging modalities.

The controller 104 prioritizes certain advanced visualization techniques based on the user's ultimate goals. In some cases, the controller 104 prioritizes outputting a smooth and high-definition color video stream to the user above other advanced visualization techniques. In other cases, the controller 104 prioritizes one or more advanced visualization techniques over color visualization, and in these cases, the output color video stream may appear choppy to a human eye because the system outputs fewer than 30 fps of color imaging data.

For example, a user may indicate that a fluorescent reagent has been administered to a patient. If the fluorescent reagent is time sensitive, then the controller 104 may ensure that a sufficient ratio of frames is devoted to fluorescence imaging to ensure the user receives adequate fluorescence imaging data while the reagent remains active. In another example, a user requests a notification whenever the user's tool comes within a threshold distance of a certain tissue, such as a blood vessel, nerve fiber, cancer tissue, and so forth. In this example, the controller 104 may prioritize laser mapping visualization to constantly determine the distance between the user's tool and the surrounding structures and may further prioritize multispectral or fluorescence imaging that enables the system to identify the certain tissue. The controller 104 may further prioritize color visualization to ensure the user continues to view a color video stream of the scene.

FIGS. 3A-3C illustrate schematic diagrams of a system 300 for processing data output by an image sensor 124 comprising the pixel array 125. The system 300 includes a controller 104 in communication with each of the emitter 102 and the image sensor 124 comprising the pixel array 125. The emitter 102 includes one or more visible sources 304, multispectral waveband sources 306, fluorescence waveband sources 308, and mapping pattern sources 310 of EMR.

The pixel array data readout 342 of the image sensor 124 includes one or more of color imaging data 305, multispectral imaging data 307, fluorescence imaging data 309, or mapping data 311. The color imaging data 305 may include one or more of a color data frame 205 captured in a time-division system configuration as illustrated in FIGS. 2A-2C or color imaging data captured with a color filter array (CFA) or multispectral filter array (MSFA). The multispectral imaging data 307 may include one or more of a multispectral data frame 207 captured in a time-division system configuration as illustrated in FIGS. 2A-2C or multispectral imaging data captured with a MSFA. The fluorescence imaging data 309 may include one or more of a fluorescence data frame 209 captured in a time-division system configuration as illustrated in FIGS. 2A-2C or fluorescence imaging data captured with a MSFA. The mapping data 311 may include one or more of a mapping data 211 or mapping data calculated with stereoscopic imaging.

When the pixel array 125 is equipped with a MSFA, the color imaging data 305 may be captured simultaneously with one or more of the multispectral imaging data 307, the fluorescence imaging data 309, or the mapping data 311. These data types may be captured simultaneously according to the time-division system configuration discussed in connection with FIGS. 2A-2C, or with a constant illumination system configuration. The type of data extracted from the pixel array data readout 342 will be depending on which EMR sources 134, 138 are cycled on by the emitter 102 during. The emitter 102 may selectively actuate any of visible sources 304, multispectral waveband sources 306, fluorescence waveband sources 308, or mapping pattern sources 310.

When data is captured according to the time-division configuration of FIGS. 2A-2C, the emitter 102 may be instructed to simultaneously cycle on the white EMR source 134 and one or more other EMR sources 138 during a blanking period of the image sensor 124. The one or more other EMR sources 138 may be tuned to emit only EMR within a narrow waveband selected for fluorescence or multispectral visualization. In this configuration, the pixel array 125 with the MSFA may simultaneously capture color visualization data and fluorescence/multispectral visualization data during a single frame period (i.e., a readout period and a blanking period).

In an alternative implementation, the emitter 102 continuously emits one or more EMR sources, including the white EMR source 134 or any of the narrowband EMR sources 138. The emitter 102 may cycle various EMR sources 134, 138 on and off based on user preferences and which datatypes are sought (i.e., color imaging data 305, multispectral imaging data 307, fluorescence imaging data 309, mapping data 311). In this implementation, the pixel array 125 equipped with the MSFA may simultaneously capture color visualization data and fluorescence/multispectral visualization data during each frame period.

As illustrated in FIG. 3B, all data read out by the pixel array may undergo frame correction 344 processing by the image signal processor 140. In various implementations, one or more of the color imaging data 305, the multispectral imaging data 307, the fluorescence imaging data 309, and the mapping data 311 undergoes frame correction 344 processes. The frame correction 344 includes one or more of sensor correction, white balance, color correction, or edge enhancement.

The multispectral imaging data 307 may undergo spectral processing 346 that is executed by the image signal processor 140 and/or another processor that is external to the system 300. The spectral processing 346 may include a machine learning algorithm and may be executed by a neural network configured to process the multispectral imaging data 307 to identify one or more tissue structures within a scene based on whether those tissue structures emitted a spectral response. The spectral processing 346 assesses the pixel integration (accumulation) values for each pixel within the pixel array 125 and may specifically assess the pixel integration values for pixels equipped with an appropriate spectral filter. The pixel integration values will inform the spectral processing 346 algorithm whether a certain pixel likely accumulated a spectral response for a certain tissue, disease, condition, chemical process, biological process, and so forth.

The fluorescence imaging data 309 may undergo fluorescence processing 348 that is executed by the image signal processor 140 and/or another processor that is external to the system 300. The fluorescence processing 348 may include a machine learning algorithm and may be executed by a neural network configured to process fluorescence imaging data 309 and identify an intensity map wherein a fluorescence relaxation wavelength is detected by the pixel array. The fluorescence processing 348 assesses the pixel integration (accumulation) values for each pixel within the pixel array 125 and may specifically assess the pixel integration values for pixels equipped with an appropriate spectral filter. The pixel integration values will inform the fluorescence processing 348 algorithm whether a certain pixel likely accumulated a fluorescence relaxation emission by a reagent or tissue.

The mapping data 311 may undergo topographical processing 350 that is executed by the image signal processor 140 and/or another processor that is external to the system 300. The topographical processing 350 may include a machine learning algorithm and may be executed by a neural network configured to assess time-of-flight information to calculate a depth map representative of the scene. The topographical processing 350 includes calculating one or more of a three-dimensional topographical map of the scene, a dimension of one or more objects within the scene, a distance between two or more objects within the scene, a distance between a tool and a certain tissue structure within the scene, and so forth.

The topographical processing 350 may additionally or alternatively be based on stereoscopic visualization. The topographical processing 350 may execute stereo imaging triangulation to calculate three-dimensional coordinates of points in a scene using two or more data frames captured from different viewpoints. This is calculated based on the principle of triangulation, which includes measuring relative positions and angles of image sensor 124 viewpoints using the resulting parallax information to determine the depth or distance of objects within a scene. In this case, the topographical processing 350 may include correspondence matching of features in two or more images, disparity estimation, depth calculation, and three-dimensional reconstruction.

FIG. 3C illustrates a schematic diagram of a system 300 and process flow for managing data output at an irregular rate. The image sensor 124 operates according to a sensor cycle that includes blanking periods and readout periods. The image sensor 124 outputs a data frame at the conclusion of each readout period that includes an indication of the amount of EMR the pixel array accumulated during the preceding accumulation period or blanking period.

Each frame period in the sensor cycle is adjustable on a frame-by-frame basis to optimize the output of the image sensor and compensate for the pixel array 125 having varying degrees of sensitivity to different wavebands of EMR. The duration of each blanking period may be shortened or lengthened to customize the amount of EMR the pixel array 125 can accumulate. Additionally, the duration of each readout period may be shortened or lengthened by implementing a pixel binning configuration or causing the image sensor to read out each pixel within the pixel array 125. Thus, the image sensor 124 may output data frames at an irregular rate due to the sensor cycle comprising a variable frame rate. The system 300 includes a memory buffer 352 that receives data frames from the image sensor 124. The memory buffer 352 stores the data frames and then outputs each data frame to the image signal processor 140 at a regular rate. This enables the image signal processor 140 to process each data frame in sequence at a regular rate.

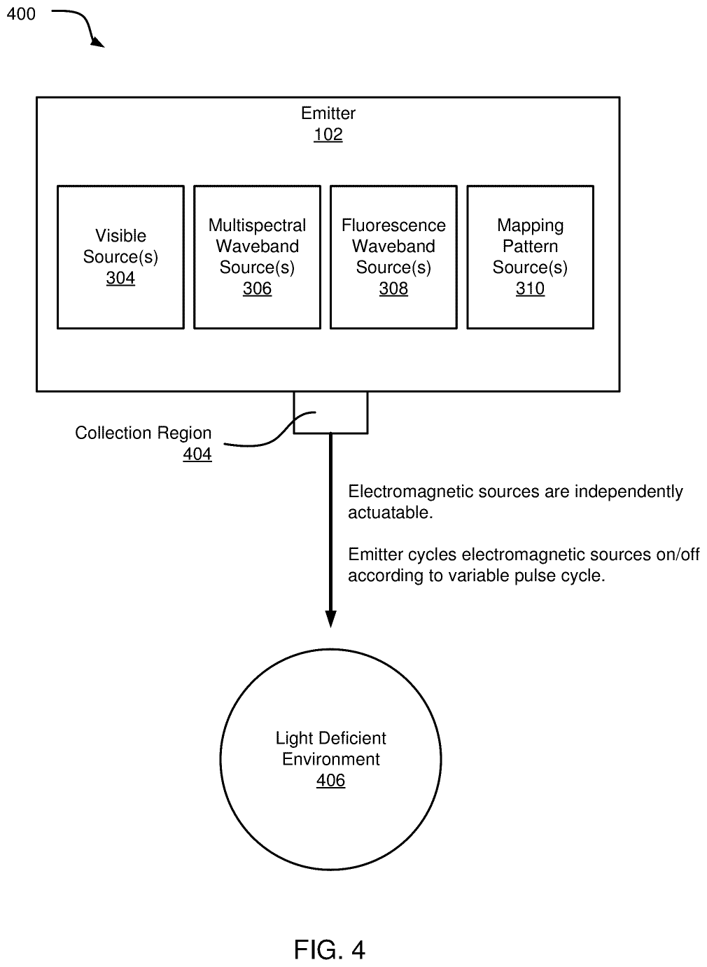

FIG. 4 is a schematic diagram of an illumination system 400 for illuminating a light deficient environment 406 such as an interior of a body cavity. In most cases, the emitter 102 is the only source of illumination within the light deficient environment 406 such that the pixel array of the image sensor does not detect any ambient light sources. The emitter 102 includes a plurality of separate and independently actuatable sources of EMR, which may include visible source(s) 304, multispectral waveband source(s) 306, fluorescence waveband source(s) 308, and mapping pattern source(s) 310. The emitter may cycle a selection of the sources on and off to pulse according to the variable pulse cycle received from the controller 104. Each of the EMR sources feeds into a collection region 404 of the emitter 102. The collection region 404 may then feed into a waveguide (see e.g., 130 in FIG. 1A) that transmits the pulsed EMR to a distal end of an endoscope within the light deficient environment 406.

The variable pulsing cycle is customizable and adjustable in real-time based on user input. The emitter 102 may instruct the individual EMR sources to pulse in any order. Additionally, the emitter 102 may adjust one or more of a duration or an intensity of each pulse of EMR. The variable pulse cycle may be optimized to sufficiently illuminate the light deficient environment 406 such that the resultant data frames read out by the pixel array 125 are within a desired exposure range (i.e., the frames are neither underexposed nor overexposed). The desired exposure range may be determined based on user input, requirements of the image signal processor 140, and/or requirements of a certain image processing algorithm (see 344, 346, 348, and 350 in FIG. 3B). The sufficient illumination of the light deficient environment 406 is dependent on the energy output of the individual EMR sources and is further dependent on the efficiency of the pixel array 125 for sensing different wavebands of EMR.

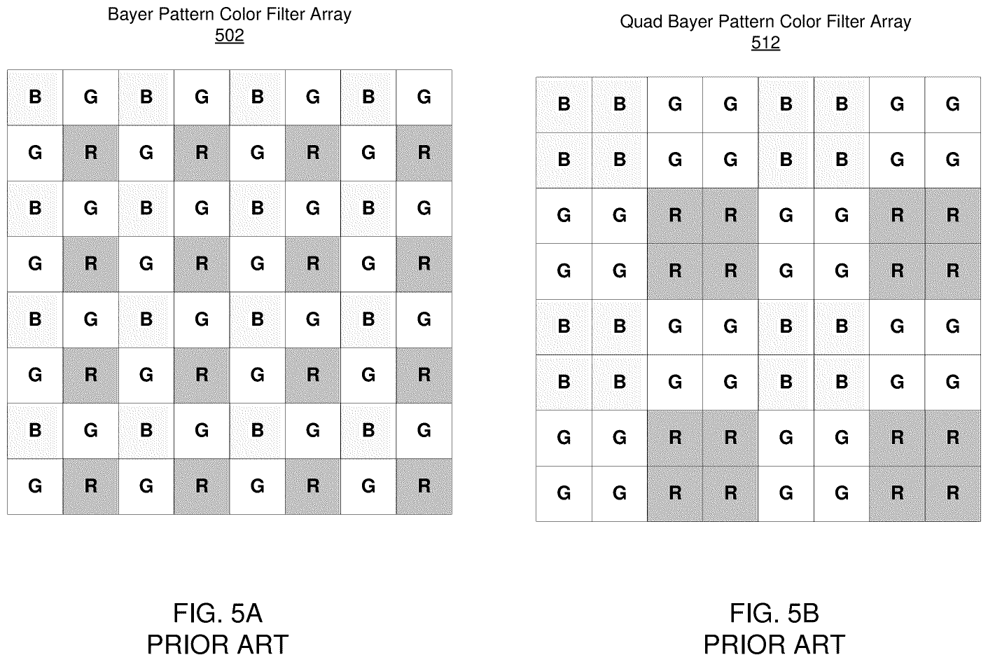

FIGS. 5A and 5B are schematic illustrations of example filter configurations for a pixel array 125 of an image sensor 124. FIG. 5A depicts a Bayer pattern color filter array 502 and FIG. 5B depicts a Quad Bayer pattern color filter array 512. The color filter arrays (CFAs) illustrated in FIGS. 5A and 5B include color filters for red, green, and blue wavebands of EMR. Thus, the CFAs are utilized to simultaneously capture red, green, and blue imaging data. In some implementations, the pixel array 125 described herein may be equipped with the Bayer pattern CFA 502 or the Quad Bayer pattern CFA 512.

The Bayer pattern CFA 502 consists of a pattern of red (“R”), green (“G”), and blue (“B”) filters arranged in a repeating pattern of 2×2 pixels. Each 2×2 square includes one pixel with a red filter, one pixel with a blue filter, and two pixels with a green filter as shown in FIG. 5A. The arrangement of these filters aids in capturing color information by filtering incoming EMR that irradiates the pixel array 125. When EMR passes through a CFA, each pixel of the pixel array 125 accumulates EMR within one waveband of the electromagnetic spectrum. The missing color information is then interpolated or reconstructed using neighboring pixels that capture the other color wavebands.

The quad Bayer pattern CFA 512 similarly includes pixels equipped with red (“R”), green (“G”), or blue (“B”) filters. However, the quad Bayer pattern CFA 512 is based on a repeating pattern of 4×4 pixels. Each 4×4 square includes four pixels with a red filter, four pixels with a blue filter, and eight pixels with a green filter as shown in FIG. 5B.

The CFAs 502, 512 may be utilized to capture color, fluorescence, multispectral, and/or laser mapping visualization data in a time sequenced manner as described herein (see, e.g., FIGS. 2A-2C). However, standard CFAs like those illustrated in FIGS. 5A-5B are not ideal for use in spectral visualization because the red, green, and blue filters are not optimized for transmitting certain wavebands of EMR selected for spectral imaging. The red, green, and blue filters may impede the pixel array's 125 efficiency in detecting multispectral or fluorescence visualization data within the visible and near infrared spectrums, but these wavebands may still be detected without using waveband-specific filters or a monochromatic (color agnostic) pixel array 125. In some cases, it is desirable to utilize a multispectral filter array (MSFA) for capturing multispectral or fluorescence visualization data.

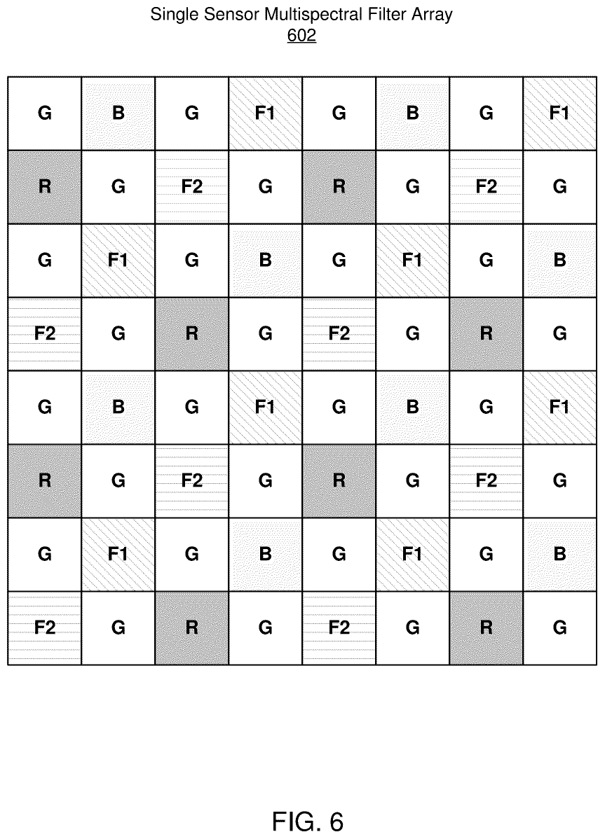

FIG. 6 is a schematic illustration of an example filter configuration for a pixel array 125 of an image sensor 124, and specifically illustrates an example single sensor multispectral filter array 602. A multispectral filter array (MSFA) is an advanced filter array used to capture image data in multiple spectral bands or wavelengths of the electromagnetic spectrum. Unlike the Bayer pattern CFAs 502, 512, which are tuned to capture only red, green, and blue color components, the MSFA incorporates filters that allow the pixel array 125 to accumulate EMR from other wavebands across a broader range of the electromagnetic spectrum, and/or to capture spectral reflectance emissions within narrow wavebands of the electromagnetic spectrum.

Typically, an MSFA includes a repeating grid of filters, wherein each filter is designed to allow EMR within specific wavebands to pass through the filter and irradiate the corresponding pixel of the pixel array 125. The arrangement of filters depends on the specific application and the desired spectral wavebands to be captured. The filters may be tuned to allow narrow or wide wavebands of EMR to pass through. The MSFA may be optimized to enable the image sensor 124 to simultaneously capture two or more of color imaging data 305, multispectral imaging data 307, fluorescence imaging data 309, or mapping data 311. The resultant data frames thus provide more detailed information about the scene being captured. By capturing data frames in multiple spectral bands simultaneously, the MSFA allows the analysis objects and materials based on their spectral responses in different wavelength ranges. This enables the identification and differentiation of various materials, the detection of specific characteristics, and the extraction of valuable information that may not be visible in standard color imaging data 305. Additionally, use of the MSFA reduces the total capture time for retrieving data across multispectral spectral wavebands and reduces or eliminates the need to account for movement when capturing spectral data across multiple frames.

Any of the MSFAs described herein may be implemented within the system 100 for endoscopic visualization of a scene. Each MSFA described herein includes a plurality of filters, and each of the plurality of filters is disposed in front of one or more pixels of the pixel array 125 (i.e., EMR passes through the filter prior to being accumulated by the one or more pixels). Each filter is configured to transmit certain wavelengths of EMR and block or filter out other wavelengths of EMR. For example, a red filter is configured to transmit red EMR (i.e., allow red EMR to pass through to the one or more pixels disposed behind the filter) and filter out or block other wavelengths of EMR from reaching the one or more pixels disposed behind the filter. Further for example, a spectral filter may be configured to transmit only EMR within a narrow waveband that corresponds with a spectral reflectance waveband of a tissue. In this case, the spectral filter will allow only the spectral reflectance to pass through to the one or more pixels disposed behind the spectral filter and will filter out or block other wavelengths of EMR.

The MSFAs described herein enable the image sensor 124 to simultaneously output color visualization data and multispectral visualization data. An image sensor 124 equipped with a MSFA as described herein will output pixel integration values for pixels equipped with a red, green, or blue filter, and these pixel integration values will be utilized to generate color visualization data for the scene. Additionally, an image sensor 124 equipped with a MSFA as described herein will simultaneously output pixel integration values for pixels equipped a spectral waveband filter, and these pixel integration values will be utilized to generate multispectral visualization data for the scene. The spectral waveband filters of the MSFA may be selected to correspond with the known spectral reflectance waveband of one or more components within a scene (i.e., the wavelengths of EMR the one or more components are known to reflect).

The example single sensor MSFA 602 may be implemented in a system 100 that comprises only one image sensor 124. Alternatively, the single MSFA 602 may be implemented in a system 100 comprises multiple image sensors 124, and two or more of the multiple image sensors 124 may be equipped with the same single MSFA 602. The single sensor MSFA 602 includes pixels equipped with red filters (“R”), green filter (“G”), blue filters (“B”), first spectral filters (“F1”), and second spectral filters (“F2”). The first spectral filters may be tuned to allow a different waveband of EMR to pass through when compared with the second spectral filters. The first and second spectral filters may each be tuned to allow a certain narrowband of EMR to pass through an irradiate the corresponding pixel. The narrow bands of EMR may be selected depending on the requested data type(s).

The example single sensor MSFA 602 includes a repeating 4×4 grid of pixels. A single 4×4 grid includes two pixels equipped with a red filter, eight pixels equipped with a green filter, two pixels equipped with a blue filter, two pixels equipped with a first spectral filter, and two pixels equipped with a second spectral filter.

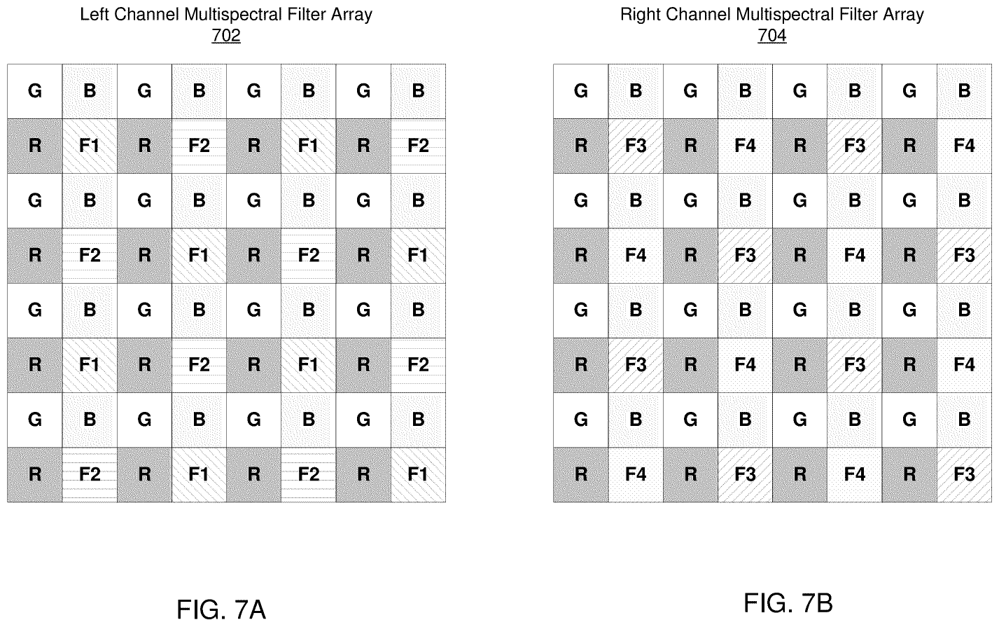

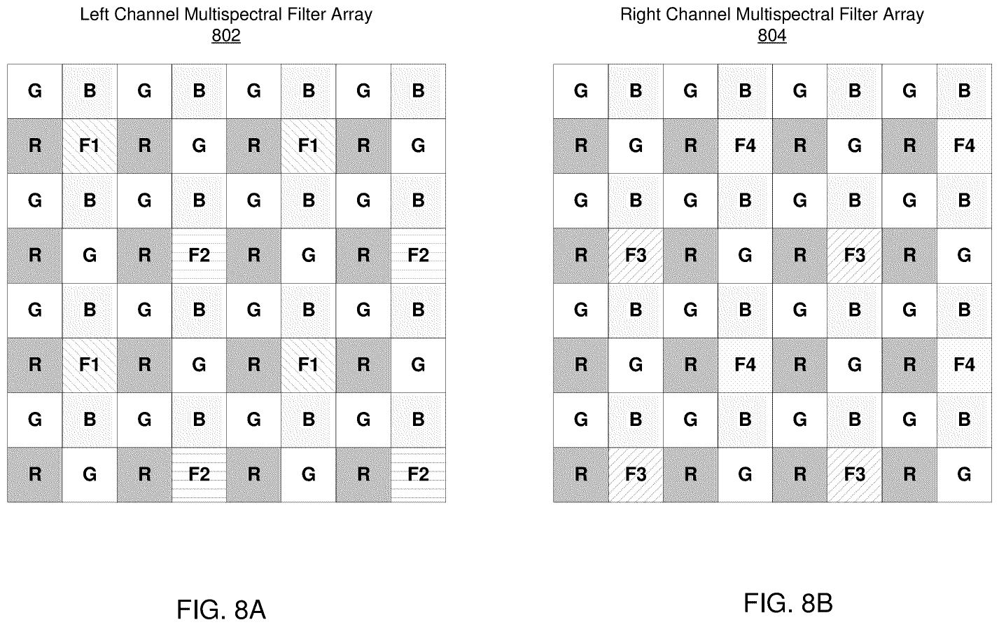

The single sensors MSFA 602 provides numerous benefits over standard CFAs like those illustrated in FIGS. 5A-5B. The wavelength-specific filter bands (F1 and F2) allow for multiple EMR sources 134, 138 to be simultaneously cycled on without causing cross-contamination of light. This speeds up the detection rates and improves the recorded light intensity in the resultant data frames. However, the single sensor MSFA 602 is limited in how many pixels may be devoted to spectral wavebands because it is important to ensure the image sensor 124 continues to capture a sufficient resolution of red, green, and blue image data. When utilizing a stereoscopic camera with two or more image sensors 124, it can be desirable to utilize different MSFAs with different spectral filter bands in each image sensor 124. This is illustrated in FIGS. 7A-7B and 8A-8B.

FIGS. 7A and 7B are schematic illustrations of an example pairing of filter configurations for a stereoscopic pair of image sensors 124. FIG. 7A is a schematic illustration of a left channel multispectral filter array (MSFA) 702 and FIG. 7B is a schematic illustration of a right channel MSFA 704. Thus, the system 100 may be equipped with two image sensors 124, and the pixel array 125 of each image sensor 124 may be equipped with a different MSFA.

Stereo image sensors 124 equipped with the different MSFAs 702, 704 may simultaneously capture different imaging data. The simultaneous imaging data may be assessed to generate a three-dimensional image comprising color imaging data 305 and one or more of multispectral imaging data 307 or fluorescence imaging data 309. The data output by the stereo image sensors 124 with different MSFAs may be utilized to output a video stream with high spatial resolution and high color resolution.

The left channel MSFA 702 includes pixels equipped with red filters (“R”), green filter (“G”), blue filters (“B”), first spectral filters (“F1”), and second spectral filters (“F2”). The example left channel MSFA 702 includes a 4×4 grid of pixels such that each 4×4 grid includes four pixels with a red filter, four pixels with a green filter, four pixels with a blue filter, two pixels with a first spectral filter, and two pixels with a second spectral filter.

The right channel MSFA 704 includes pixels equipped with red filters (“R”), green filter (“G”), blue filters (“B”), third spectral filters (“F3”), and fourth spectral filters (“F4”). The example right channel MSFA 704 includes a 4×4 grid of pixels such that each 4×4 grid includes four pixels with a red filter, four pixels with a green filter, four pixels with a blue filter, two pixels with a third spectral filter, and two pixels with a fourth spectral filter.

Collectively, the left image sensor 124 equipped with the left channel MSFA 702 and the right image sensor 124 equipped with the right channel MSFA 704 will simultaneously output color imaging data and four different types of spectral imaging data. The spectral imaging data may include one or more of multispectral imaging data 307 or fluorescence imaging data 309. The type(s) of spectral imaging data will depend on the wavebands of EMR allowed to pass through each of the first, second, third, and fourth spectral filters (F1, F2, F3, F4).

FIGS. 8A and 8B are schematic illustrations of an example pairing of filter configurations for a stereoscopic pair of image sensors 124. The example pairing illustrated in FIGS. 8A-8B is an alternative to the pairing illustrated in FIGS. 7A-7B. The pairing illustrated in FIGS. 8A-8B may be selected when it is desirable to extract additional green color imaging data. FIG. 8A is a schematic illustration of a left channel multispectral filter array (MSFA) 802 and FIG. 8B is a schematic illustration of a right channel MSFA 804.

The left channel MSFA 802 includes pixels equipped with red filters (“R”), green filter (“G”), blue filters (“B”), first spectral filters (“F1”), and second spectral filters (“F2”). The example left channel MSFA 802 includes a 4×4 grid of pixels such that each 4×4 grid includes four pixels with a red filter, six pixels with a green filter, four pixels with a blue filter, one pixel with a first spectral filter, and one pixel with a second spectral filter.

The right channel MSFA 804 includes pixels equipped with red filters (“R”), green filter (“G”), blue filters (“B”), third spectral filters (“F3”), and fourth spectral filters (“F4”). The example right channel MSFA 804 includes a 4×4 grid of pixels such that each 4×4 grid includes four pixels with a red filter, six pixels with a green filter, four pixels with a blue filter, one pixel with a third spectral filter, and one pixel with a fourth spectral filter.

The multispectral filter arrays illustrated in FIGS. 6, 7A-7B, and 8A-8B are exemplary only, and other MSFAs may be implemented in the system 100 without departing from the scope of the disclosure. The MSFAs may include any suitable number of unique spectral filters depending on the implementation and the desired visualization result. In the example illustrated in FIGS. 7A-7B and 8A-8B, the image sensors 124 will collectively output spectral visualization data associated with four unique spectral wavebands. However, in other implementations, the image sensors 124 may collectively output spectral visualization data associated with fewer or more unique spectral wavebands, such as one, two, three, five, six, seven, eight, nine, or ten unique spectral wavebands. The present disclosure does not limit the quantity of unique spectral waveband filters included in the MSFAs of the stereoscopic camera.

In some implementations, each MSFA is optimized for visualizing a certain tissue, biological process, reagent, chemical process, or condition. For example, a left channel MSFA might include spectral filters selected to transmit wavelengths of EMR associated with the spectral response of venous tissue, and the accompanying right channel MSFA might include spectral filters selected to transmit wavelengths of EMR associated with the spectral response of arterial tissue. The spectral filters may be selected based on what types of tissues or conditions will be identified by the system 100. Each spectral filter may be configured to transmit a waveband of EMR that is known to be associated with the spectral response emitted by a certain tissue, biological process, reagent, chemical process, or condition.

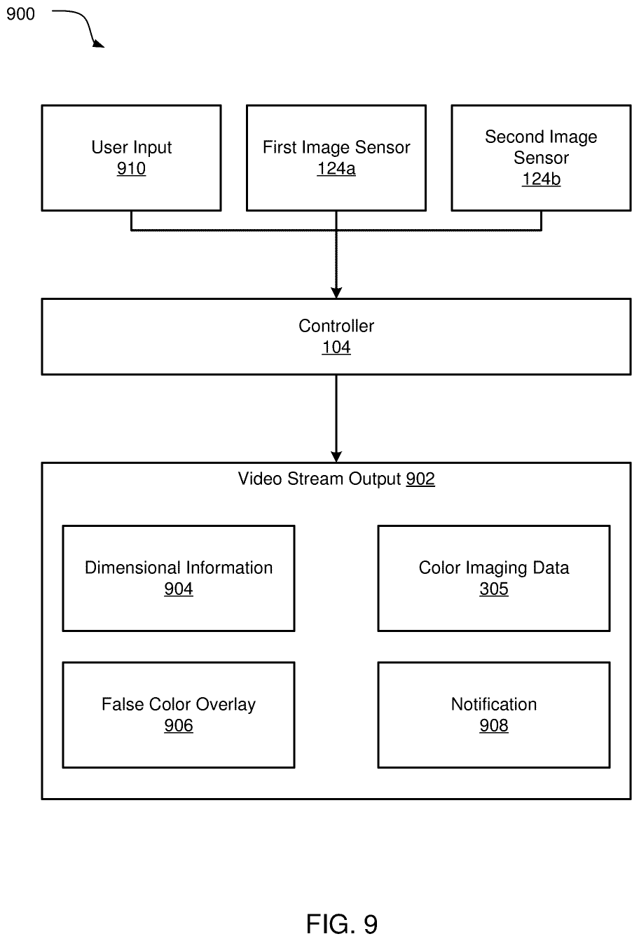

FIG. 9 is a schematic diagram of an example process flow 900 for image signal processing of data output by a stereoscopic camera as described herein. The process flow 900 is executed by the controller 104 of the endoscopic visualization system 100. The controller 104 receives imaging data from a first image sensor 124a and a second image sensor 124 of a stereoscopic camera of an endoscope. The controller 104 additionally receives a user input 910, which may include a request that a certain type of tissue, chemical process, or biological process be identified by the system 100. The controller renders a video stream output 902 based on the stereoscopic camera imaging data and the user input 910. The video stream output 902 includes one or more of dimensional information 904, color imaging data 305, a false color overlay 906, or a notification 908.

Each of the first image sensor 124a and the second image sensor 124b may be equipped with a multispectral filter array (MSFA). In an implementation, a first MSFA of the first image sensor 124a is different from a second MSFA of the second image sensor 124b. Each of the first MSFA and the second MSFA may include red, green, and blue color filters such that each of the first image sensor 124a and the second image sensor 124b can output color imaging data 305. However, the first MSFA and the second MSFA may be equipped with different spectral filters that are configured to transmit different wavebands of EMR.

The controller 104 calculates the dimensional information 904 by triangulating data output by the first image sensor 124a and the second image sensor 124b. The first image sensor 124a and the second image sensor 124b may each simultaneously output a data frame. Because the first image sensor 124a and the second image sensor 124b are located at slightly different positions (see, e.g., FIG. 1B), the data frames output by the first and second image sensors 124a, 124b will be captured from slightly different angles. The controller 104 combines the two slightly different views captured in the pair of data frames and interprets the differences as depth information.

The controller 104 may execute various image processing and image optimization algorithms when rendering the color imaging data 305 for a display. The color imaging data 305 may be rendered as a three-dimensional image by combining data output by the first and second image sensors 124a, 124b. In most implementations, a singular color image frame is rendered based on stereoscopic data simultaneously output by the first and second image sensors 124a, 124b. This data is output by the red, green, and blue-filtered pixels of each of the first and second image sensors 124a, 124b, and is then processed to be rendered on a display. In other cases, only one of the two image sensors 124a, 124b outputs data for the color imaging data 305. Further, the image sensors 124a, 124b may be instructed to output image data in a time-division manner such that the image sensors 124a, 124b are not simultaneously outputting data frames.

The controller 104 renders the false color overlay 906 to comply with a user input 910. In an example implementation, the user input 910 may include a request to render a false color overlay that highlights a certain tissue structure within a scene, such as a vein, artery, ureter tissue, nervous tissue, cardiovascular tissue, cancerous tissue, and so forth. The user input 910 might alternatively or additionally request that the controller 104 renders a false color overlay highlighting the location of a chemical process, biological process, fluorescence reagent, or auto fluorescing tissue. The false color overlay 906 is overlaid on the color imaging data 305 to generate an overlay frame, and then the overlay frame is provided to a display.

The controller 104 extracts information for the false color overlay 906 based on data frames output by the image sensors 124a, 124b. In most cases, the false color overlay 906 is generated based on advanced visualization data such as multispectral imaging data 307 or fluorescence imaging data 309. The multispectral imaging data 307 and the fluorescence imaging data 309 may simultaneously be output along with the color imaging data 305 due to the MSFAs equipped on the image sensors 124a, 124b.

The controller 104 generates a notification 908 to comply with a user input 910 or system setting. The notification 908 might include, for example, an indication that a tool is within a threshold distance of a tissue, an indication that a critical tissue structure is present within the scene, an indication that two or more tools are within a threshold distance from each other, and so forth. The notification may include a visual, auditory, or tactical notification. In some cases, the notification includes an overlay rendered over color imaging data 305 depicting a scene. In these cases, the overlay may include an alert or alphanumerical message.

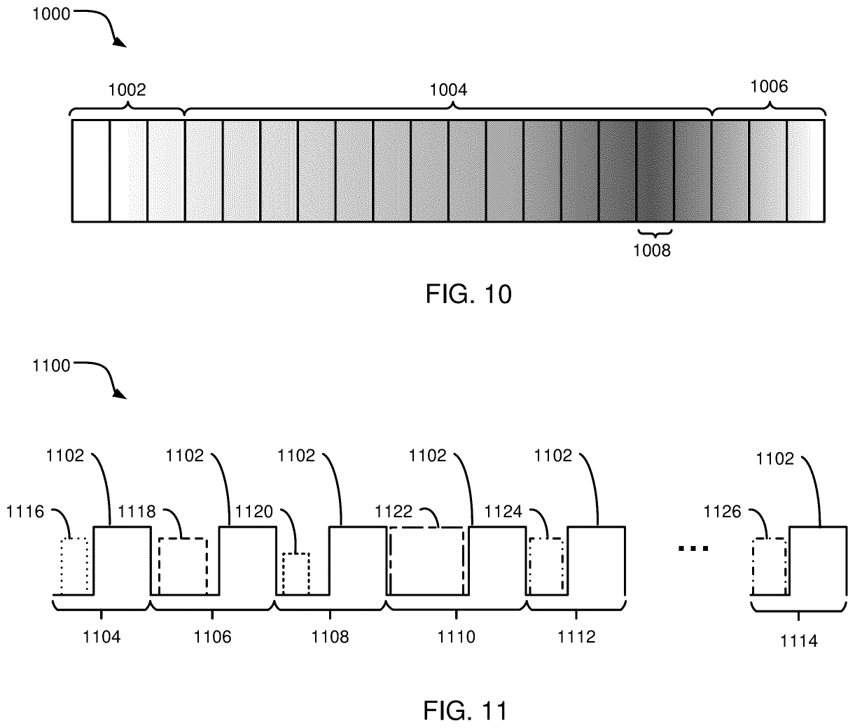

FIG. 10 illustrates a portion of the electromagnetic spectrum 1000 divided into twenty different wavebands. The number of wavebands is illustrative only. In at least one embodiment, the spectrum 1000 may be divided into hundreds of wavebands. The spectrum 1000 may extend from the infrared spectrum 1002, through the visible spectrum 1004, and into the ultraviolet spectrum 1006. In some cases, it can be particularly useful to capture spectral response data in the near infrared spectrum, which constitutes a narrow portion of the wide infrared spectrum 1002. Each waveband may be defined by an upper wavelength and a lower wavelength.

The MSFAs described herein may be equipped with customized spectral filters configured to transmit EMR within any suitable waveband. It should be understood that the wavebands of the spectral filters will be selected based on the intended use-case for the system 100 and may specifically be dependent on the spectral signatures of tissues to be identified with the system 100.

Multispectral imaging incudes imaging information from across the electromagnetic spectrum 1000. A multispectral pulse of EMR may include a plurality of sub-pulses spanning one or more portions of the electromagnetic spectrum 1000 or the entirety of the electromagnetic spectrum 1000. A multispectral pulse of EMR may include a single partition of wavelengths of EMR. A resulting multispectral data frame includes information sensed by the pixel array subsequent to a multispectral pulse of EMR. Therefore, a multispectral data frame may include data for any suitable partition of the electromagnetic spectrum 1000 and may include multiple data frames for multiple partitions of the electromagnetic spectrum 1000.

The emitter 102 may include any number of multispectral EMR sources as needed depending on the implementation. In one embodiment, each multispectral EMR source covers a spectrum covering 40 nanometers. For example, one multispectral EMR source may emit EMR within a waveband from 500 nm to 540 nm while another multispectral EMR source may emit EMR within a waveband from 540 nm to 580 nm. In another embodiment, multispectral EMR sources may cover other sizes of wavebands, depending on the types of EMR sources available or the imaging needs. Each multispectral EMR source may cover a different slice of the electromagnetic spectrum 1000 ranging from far infrared, mid infrared, near infrared, visible light, near ultraviolet and/or extreme ultraviolet. In some cases, a plurality of multispectral EMR sources of the same type or wavelength may be included to provide sufficient output power for imaging. The number of multispectral EMR sources needed for a specific waveband may depend on the sensitivity of a pixel array 125 to the waveband and/or the power output capability of EMR sources in that waveband.

The waveband widths and coverage provided by the EMR sources may be selected to provide any desired combination of spectrums. For example, contiguous coverage of a spectrum 1000 using small waveband widths (e.g., 10 nm or less) may allow for highly selective multispectral and/or fluorescence imaging. The waveband widths allow for selectively emitting the excitation wavelength(s) for one or more particular fluorescent reagents. Additionally, the waveband widths may allow for selectively emitting certain partitions of multispectral EMR for identifying specific structures, chemical processes, tissues, biological processes, and so forth. Because the wavelengths come from EMR sources which can be selectively activated, extreme flexibility for fluorescing one or more specific fluorescent reagents during an examination can be achieved. Additionally, extreme flexibility for identifying one or more objects or processes by way of multispectral imaging can be achieved. Thus, much more fluorescence and/or multispectral information may be achieved in less time and within a single examination which would have required multiple examinations, delays because of the administration of dyes or stains, or the like.

FIG. 11 is a schematic diagram illustrating a timing diagram 1100 for emission and readout for generating an image. The solid line represents readout (peaks 1102) and blanking periods (valleys) for capturing a series of data frames 1104-1114. The series of data frames 1104-1114 may include a repeating series of data frames which may be used for generating mapping, multispectral, and/or fluorescence data that may be overlaid on an RGB video stream. The series of data frames include a first data frame 1104, a second data frame 1106, a third data frame 1108, a fourth data frame 1110, a fifth data frame 1112, and an Nth data frame 1114.

In one embodiment, each data frame is generated based on at least one pulse of EMR. The pulse of EMR is reflected and detected by the pixel array 125 and then read out in a subsequent readout (1102). Thus, each blanking period and readout results in a data frame for a specific waveband of EMR. For example, the first data frame 1104 may be generated based on a waveband of a first one or more pulses 1116, a second data frame 1106 may be generated based on a waveband of a second one or more pulses 1118, a third data frame 1108 may be generated based on a waveband of a third one or more pulses 1120, a fourth data frame 1110 may be generated based on a waveband of a fourth one or more pulses 1122, a fifth data frame 1112 may be generated based on a waveband of a fifth one or more pulses 1124, and an Nth data frame 1114 may be generated based on a waveband of an Nth one or more pulses 1126.

The pulses 1116-1126 may include energy from a single EMR source or from a combination of two or more EMR sources. For example, the waveband included in a single readout period or within the plurality of data frames 1104-1114 may be selected for a desired examination or detection of a specific tissue or condition. According to one embodiment, one or more pulses may include visible spectrum light for generating an RGB or black and white image while one or more additional pulses are emitted to sense a spectral response to a multispectral wavelength of EMR.

The pulses 1116-1126 are emitted according to a variable pulse cycle determined by the controller 104. For example, pulse 1116 may include a white light, pulse 1118 may include a multispectral waveband, pulse 1120 may include a white light, pulse 1122 may include a fluorescence waveband, pulse 1124 may include white light, and so forth.

The plurality of frames 1104-1114 are shown having varying lengths in readout periods and pulses having different lengths or intensities. The blanking period, pulse length or intensity, or the like may be selected based on the sensitivity of a monochromatic sensor to the specific wavelength, the power output capability of the EMR source(s), and/or the carrying capacity of the waveguide.

In one embodiment, dual image sensors may be used to obtain three-dimensional images or video feeds. A three-dimensional examination may allow for improved understanding of a three-dimensional structure of the examined region as well as a mapping of the different tissue or material types within the region.

In an example implementation, a patient is imaged with an endoscopic imaging system to identify quantitative diagnostic information about the patient's tissue pathology. In the example, the patient is suspected or known to suffer from a disease that can be tracked with multispectral imaging to observe the progression of the disease in the patient's tissue. The endoscopic imaging system pulses white light to generate an RGB video stream of the interior of the patient's body. Additionally, the endoscopic imaging system pulses one or more multispectral wavebands of light that permit the system to “see through” some tissues and generate imaging of the tissue affected by the disease. The endoscopic imaging system senses the reflected multispectral EMR to generate multispectral imaging data of the diseased tissue, and thereby identifies the location of the diseased tissue within the patient's body. The endoscopic imaging system may further emit a mapping pulsing scheme for generating a three-dimensional topographical map of the scene and calculating dimensions of objects within the scene. The location of the diseased tissue (as identified by the multispectral imaging data) may be combined with the topographical map and dimensions information that is calculated with the mapping data. Therefore, the precise location, size, dimensions, and topology of the diseased tissue can be identified. This information may be provided to a medical practitioner to aid in excising, imaging, or studying the diseased tissue. Additionally, this information may be provided to a robotic surgical system to enable the surgical system to excise the diseased tissue.

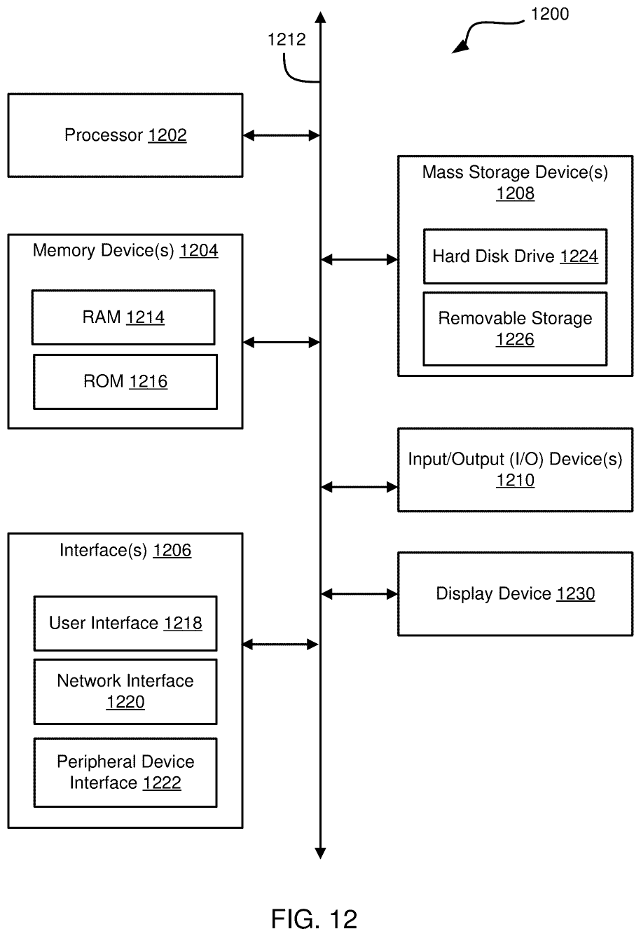

FIG. 12 illustrates a schematic block diagram of an example computing device 1200. The computing device 1200 may be used to perform various procedures, such as those discussed herein. The computing device 1200 can perform various monitoring functions as discussed herein, and can execute one or more application programs, such as the application programs or functionality described herein. The computing device 1200 can be any of a wide variety of computing devices, such as a desktop computer, in-dash computer, vehicle control system, a notebook computer, a server computer, a handheld computer, tablet computer and the like.

The computing device 1200 includes one or more processor(s) 1204, one or more memory device(s) 1204, one or more interface(s) 1206, one or more mass storage device(s) 1208, one or more Input/output (I/O) device(s) 1210, and a display device 1230 all of which are coupled to a bus 1212. Processor(s) 1204 include one or more processors or controllers that execute instructions stored in memory device(s) 1204 and/or mass storage device(s) 1208. Processor(s) 1204 may also include several types of computer-readable media, such as cache memory.

Memory device(s) 1204 include various computer-readable media, such as volatile memory (e.g., random access memory (RAM) 1214) and/or nonvolatile memory (e.g., read-only memory (ROM) 1216). Memory device(s) 1204 may also include rewritable ROM, such as Flash memory.

Mass storage device(s) 1208 include various computer readable media, such as magnetic tapes, magnetic disks, optical disks, solid-state memory (e.g., Flash memory), and so forth. As shown in FIG. 12, a particular mass storage device 1208 is a hard disk drive 1224. Various drives may also be included in mass storage device(s) 1208 to enable reading from and/or writing to the various computer readable media. Mass storage device(s) 1208 include removable media 1226 and/or non-removable media.

I/O device(s) 1210 include various devices that allow data and/or other information to be input to or retrieved from computing device 1200. Example I/O device(s) 1210 include cursor control devices, keyboards, keypads, microphones, monitors or other display devices, speakers, printers, network interface cards, modems, and the like.

Display device 1230 includes any type of device capable of displaying information to one or more users of computing device 1200. Examples of display device 1230 include a monitor, display terminal, video projection device, and the like.

Interface(s) 1206 include various interfaces that allow computing device 1200 to interact with other systems, devices, or computing environments. Example interface(s) 1206 may include any number of different network interfaces 1220, such as interfaces to local area networks (LANs), wide area networks (WANs), wireless networks, and the Internet. Other interface(s) include user interface 1218 and peripheral device interface 1222. The interface(s) 1206 may also include one or more user interface elements 1218. The interface(s) 1206 may also include one or more peripheral interfaces such as interfaces for printers, pointing devices (mice, track pad, or any suitable user interface now known to those of ordinary skill in the field, or later discovered), keyboards, and the like.

Bus 1212 allows processor(s) 1204, memory device(s) 1204, interface(s) 1206, mass storage device(s) 1208, and I/O device(s) 1210 to communicate with one another, as well as other devices or components coupled to bus 1212. Bus 1212 represents one or more of several types of bus structures, such as a system bus, PCI bus, IEEE bus, USB bus, and so forth.

For purposes of illustration, programs and other executable program components are shown herein as discrete blocks, such as block 302 for example, although it is understood that such programs and components may reside at various times in different storage components of computing device 1200 and are executed by processor(s) 1202. Alternatively, the systems and procedures described herein, including programs or other executable program components, can be implemented in hardware, or a combination of hardware, software, and/or firmware. For example, one or more application specific integrated circuits (ASICs) can be programmed to carry out one or more of the systems and procedures described herein.

### Examples

The following examples pertain to preferred features of further embodiments:

Example 1 is a system. The system includes a first image sensor comprising a first pixel array, wherein the first pixel array comprises a first multispectral filter array that transmits electromagnetic radiation within a first waveband. The system includes a second image sensor comprising a second pixel array, wherein the second pixel array comprises a second multispectral filter array that transmits electromagnetic radiation within a second waveband. The system is such that the first waveband is different from the second waveband.

Example 2 is a system as in Example 1, further comprising an endoscope tube, wherein each of the first image sensor and the second image sensor is disposed within an interior cavity of the endoscope tube.

Example 3 is a system as in any of Examples 1-2, further comprising an emitter comprising a plurality of sources of electromagnetic radiation, wherein the plurality of sources comprises: a visible source that emits broadband visible electromagnetic radiation; a first spectral source that emits the first waveband of electromagnetic radiation; and a second spectral source that emits the second waveband of electromagnetic radiation.

Example 4 is a system as in any of Examples 1-3, wherein the first multispectral filter array and the second multispectral filter array further comprises: a plurality of red filters that transmit electromagnetic radiation within a red waveband; a plurality of green filters that transmit electromagnetic radiation within a green waveband; and a plurality of blue filters that transmit electromagnetic radiation within a blue waveband.

Example 5 is a system as in any of Examples 1-4, wherein, for each of the first multispectral filter array and the second multispectral array, a quantity of the plurality of green filters is greater than a quantity of either of the plurality of red filters or the plurality of blue filters.

Example 6 is a system as in any of Examples 1-5, wherein the first multispectral filter array comprises: a plurality of red filters that transmit electromagnetic radiation within a red waveband; a plurality of green filters that transmit electromagnetic radiation within a green waveband; a plurality of blue filters that transmit electromagnetic radiation within a blue waveband; a plurality of first spectral filters that transmit the electromagnetic radiation within the first waveband; and a plurality of third spectral filters that transmit electromagnetic radiation within a third spectral waveband.

Example 7 is a system as in any of Examples 1-6, wherein the second multispectral filter array comprises: a plurality of red filters that transmit the electromagnetic radiation within the red waveband; a plurality of green filters that transmit the electromagnetic radiation within the green waveband; a plurality of blue filters that transmit the electromagnetic radiation within the blue waveband; a plurality of second spectral filters that transmit the electromagnetic radiation within the second waveband; and a plurality of fourth spectral filters that transmit electromagnetic radiation within a fourth spectral waveband.

Example 8 is a system as in any of Examples 1-7, wherein the first multispectral filter array and the second multispectral filter array collectively transmit a plurality of spectral wavebands of electromagnetic radiation selected for multispectral visualization or fluorescence visualization of a scene.

Example 9 is a system as in any of Examples 1-8, wherein the plurality of spectral wavebands comprises four or more unique spectral wavebands.

Example 10 is a system as in any of Examples 1-9, wherein each of the first waveband and the second waveband is a narrowband of wavelengths selected for multispectral visualization or fluorescence visualization of a scene.

Example 11 is a system as in any of Examples 1-10, wherein each of the first waveband and the second waveband is 20 nm wide or less.

Example 12 is a system as in any of Examples 1-11, wherein at least one of the first waveband or the second waveband is within a near infrared waveband of the electromagnetic spectrum.

Example 13 is a system as in any of Examples 1-12, wherein at least one of the first waveband or the second waveband is within a visible waveband of the electromagnetic spectrum and is 20 nm wide or less.

Example 14 is a system as in any of Examples 1-13, wherein at least one of the first multispectral filter array or the second multispectral filter array comprises a tunable filter and/or a removable filter such that a user may exchange one or more of the first multispectral filter array or the second multispectral filter array.

Example 15 is a system as in any of Examples 1-14, wherein the first image sensor outputs a first data frame simultaneously with the second image sensor outputting a second data frame; wherein each of the first data frame and the second data frame comprises color imaging data; wherein the first data frame comprises first spectral imaging data associated with the first waveband; and wherein the second data frame comprises second spectral imaging data associated with the second waveband.

Example 16 is a system as in any of Examples 1-15, wherein at least one of the first spectral imaging data or the second spectral imaging data comprises pixel integration values for pixels accumulating a fluorescence relaxation emission by one or more of a fluorescent reagent or an auto fluorescing tissue.

Example 17 is a system as in any of Examples 1-16, wherein at least one of the first spectral imaging data or the second spectral imaging data comprises pixel integration values for pixels accumulating a spectral response emitted by one or more of a tissue structure, a chemical process, or a biological process.

Example 18 is a system as in any of Examples 1-17, further comprising an image signal processor in communication with the first image sensor and the second image sensor, wherein the image signal processor is configured to execute instructions comprising: calculating dimensional information based on pixel integration values for the first data frame and the second data frame, and further based on a relative position of the first pixel array and the second pixel array.

Example 19 is a system as in any of Examples 1-18, wherein the instructions executed by the image signal processor further comprise rendering a three-dimensional image of a scene based on the dimensional information.

Example 20 is a system as in any of Examples 1-19, wherein the instructions executed by the image signal processor further comprise generating an overlay frame comprising: the color imaging data; and a false color overlay rendered based on one or more of the first spectral imaging data or the second spectral imaging data; wherein the false color overlay highlights a location of one or more of a tissue structure, a chemical process, or a biological process within a scene.

Example 21 is a system. The system includes an emitter comprising a plurality of sources of electromagnetic radiation, wherein the plurality of sources comprises: a visible source that emits broadband electromagnetic radiation within a visible waveband of the electromagnetic spectrum; a first spectral source that emits electromagnetic radiation within a first spectral waveband; and a second spectral source that emits electromagnetic radiation within a second spectral waveband. The system includes a first image sensor comprising a first pixel array, wherein the first pixel array comprises a first multispectral filter array comprising a first plurality of filters, and wherein at least a portion of the first plurality of filters transmits reflected electromagnetic radiation within the first spectral waveband. The system includes a second image sensor comprising a second pixel array, wherein the second pixel array comprises a second multispectral filter array comprising a second plurality of filters, and wherein at least a portion of the second plurality of filters transmits reflected electromagnetic radiation within the second spectral waveband. The system is such that the first spectral waveband is different from the second spectral waveband.

Example 22 is a system as in Example 21, further comprising an endoscope tube, wherein each of the first image sensor and the second image sensor is disposed within an interior cavity of the endoscope tube.

Example 23 is a system as in any of Examples 21-22, wherein the first plurality of filters of the first multispectral filter array comprises: a first plurality of red filters that transmit red electromagnetic radiation; a first plurality of green filters that transmit green electromagnetic radiation; a first plurality of blue filters that transmit blue electromagnetic radiation; and a first plurality of spectral filters that transmit the reflected electromagnetic radiation within the first spectral waveband; and wherein the second plurality of filters of the second multispectral filter array comprises: a second plurality of red filters that transmit the red electromagnetic radiation; a second plurality of green filters that transmit the green electromagnetic radiation; a second plurality of blue filters that transmit the blue electromagnetic radiation; and a second plurality of spectral filters that transmit the reflected electromagnetic radiation within the second spectral waveband.

Example 24 is a system as in any of Examples 21-23, wherein at least one of the first plurality of filters of the first multispectral filter array or the second plurality of filters of the second multispectral filter array comprises a third plurality of spectral filters that transmit reflected electromagnetic radiation within a third waveband, and wherein the third waveband is different from the first spectral waveband or the second spectral waveband.

Example 25 is a system as in any of Examples 21-24, wherein one or more of: a quantity of the first plurality of green filters is greater than a quantity of either of the first plurality of red filters or the first plurality of blue filters; or a quantity of the second plurality of green filters is greater than a quantity of either of the second plurality of red filters or the second plurality of blue filters.

Example 26 is a system as in any of Examples 21-25, wherein the emitter further comprises a third spectral source that emits electromagnetic radiation within a third spectral waveband, and wherein the third spectral waveband is different from the first spectral waveband or the second spectral waveband; and wherein the first plurality of filters of the first multispectral filter array comprises: a first plurality of spectral filters that transmits the reflected electromagnetic radiation within the first spectral waveband; and a third plurality of spectral filters that transmits reflected electromagnetic radiation within the third spectral waveband.

Example 27 is a system as in any of Examples 21-26, wherein the emitter further comprises a fourth spectral source that emits electromagnetic radiation within a fourth spectral waveband, and wherein the fourth spectral waveband is different from each of the first spectral waveband, the second spectral waveband, and the third spectral waveband; and wherein the second plurality of filters of the second multispectral filter array comprises: a second plurality of spectral filters that transmits the reflected electromagnetic radiation within the second spectral waveband; and a fourth plurality of spectral filters that transmits reflected electromagnetic radiation within the fourth spectral waveband.

Example 28 is a system as in any of Examples 21-27, wherein the first multispectral filter array and the second multispectral filter array collectively transmit a plurality of spectral wavebands of electromagnetic radiation selected for multispectral visualization or fluorescence visualization of a scene.

Example 29 is a system as in any of Examples 21-28, wherein each of the plurality of spectral wavebands comprises electromagnetic radiation corresponding with a spectral reflectance waveband of a tissue; wherein the spectral reflectance waveband of the tissue comprises one or more wavelengths of electromagnetic radiation that the tissue reflects; and wherein the tissue comprises one or more of venous tissue, arterial tissue, ureter tissue, nervous tissue, cardiovascular tissue, or cancerous tissue.

Example 30 is a system as in any of Examples 21-29, wherein each of the first spectral waveband and the second spectral waveband is a narrowband of wavelengths selected for multispectral visualization or fluorescence visualization of a scene.

Example 31 is a system as in any of Examples 21-30, wherein each of the first spectral waveband and the second spectral waveband is 20 nm wide or less.

Example 32 is a system as in any of Examples 21-31, wherein at least one of the first spectral waveband or the second spectral waveband is within a near infrared waveband of the electromagnetic spectrum.

Example 33 is a system as in any of Examples 21-32, wherein at least one of the first spectral waveband or the second spectral waveband is within a visible waveband of the electromagnetic spectrum and is 20 nm wide or less.

Example 34 is a system as in any of Examples 21-33, wherein at least one of the first multispectral filter array or the second multispectral filter array comprises a tunable filter.

Example 35 is a system as in any of Examples 21-34, wherein the first image sensor outputs a first data frame simultaneously with the second image sensor outputting a second data frame; wherein each of the first data frame and the second data frame comprises color imaging data; wherein the first data frame comprises first spectral imaging data associated with the first spectral waveband; and wherein the second data frame comprises second spectral imaging data associated with the second spectral waveband.

Example 36 is a system as in any of Examples 21-35, wherein at least one of the first spectral imaging data or the second spectral imaging data comprises pixel integration values for pixels accumulating a fluorescence relaxation emission by one or more of a fluorescent reagent or an auto fluorescing tissue.

Example 37 is a system as in any of Examples 21-36, wherein at least one of the first spectral imaging data or the second spectral imaging data comprises pixel integration values for pixels accumulating a spectral reflectance that is reflected by one or more of a tissue structure, a chemical process, or a biological process.

Example 38 is a system as in any of Examples 21-37, further comprising an image signal processor in communication with the first image sensor and the second image sensor, wherein the image signal processor is configured to execute instructions comprising: calculating dimensional information based on pixel integration values for the first data frame and the second data frame, and further based on a relative position of the first pixel array and the second pixel array.

Example 39 is a system as in any of Examples 21-38, wherein the instructions executed by the image signal processor further comprise rendering a three-dimensional image of a scene based on the dimensional information.

Example 40 is a system as in any of Examples 21-39, wherein the instructions executed by the image signal processor further comprises generating an overlay frame comprising: the color imaging data; and a false color overlay rendered based on one or more of the first spectral imaging data or the second spectral imaging data; wherein the false color overlay highlights a location of one or more of a tissue structure, a chemical process, or a biological process within a scene.

It will be appreciated that various features disclosed herein provide significant advantages and advancements in the art. The following claims are exemplary of some of those features.

In the foregoing Detailed Description of the Disclosure, various features of the disclosure are grouped together in a single embodiment for the purpose of streamlining the disclosure. This method of disclosure is not to be interpreted as reflecting an intention that the claimed disclosure requires more features than are expressly recited in each claim. Rather, inventive aspects lie in less than all features of a single foregoing disclosed embodiment.

It is to be understood that any features of the above-described arrangements, examples, and embodiments may be combined in a single embodiment comprising a combination of features taken from any of the disclosed arrangements, examples, and embodiments.

It is to be understood that the above-described arrangements are only illustrative of the application of the principles of the disclosure. Numerous modifications and alternative arrangements may be devised by those skilled in the art without departing from the spirit and scope of the disclosure and the appended claims are intended to cover such modifications and arrangements.

Thus, while the disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

Further, where appropriate, functions described herein can be performed in one or more of: hardware, software, firmware, digital components, or analog components. For example, one or more application specific integrated circuits (ASICs) or field programmable gate arrays (FPGAs) can be programmed to carry out one or more of the systems and procedures described herein. Certain terms are used throughout the following description and claims to refer to particular system components. As one skilled in the art will appreciate, components may be referred to by different names. This document does not intend to distinguish between components that differ in name, but not function.

The foregoing description has been presented for the purposes of illustration and description. It is not intended to be exhaustive or to limit the disclosure to the precise form disclosed. Many modifications and variations are possible in light of the above teaching. Further, it should be noted that any or all the aforementioned alternate implementations may be used in any combination desired to form additional hybrid implementations of the disclosure.

Further, although specific implementations of the disclosure have been described and illustrated, the disclosure is not to be limited to the specific forms or arrangements of parts so described and illustrated. The scope of the disclosure is to be defined by the claims appended hereto, any future claims submitted here and in different applications, and their equivalents.

What is claimed is:

1. A system for stereoscopic visualization, the system comprising:
an emitter comprising a plurality of sources of electromagnetic radiation, wherein the plurality of sources comprises:
a visible source that emits broadband electromagnetic radiation within a visible waveband of the electromagnetic spectrum;
a first spectral source that emits electromagnetic radiation within a first spectral waveband; and
a second spectral source that emits electromagnetic radiation within a second spectral waveband;
a first image sensor comprising a first pixel array, wherein the first pixel array comprises a first multispectral filter array comprising a first plurality of filters, and wherein at least a portion of the first plurality of filters transmits reflected electromagnetic radiation within the first spectral waveband;
a second image sensor comprising a second pixel array, wherein the second pixel array comprises a second multispectral filter array comprising a second plurality of filters, and wherein at least a portion of the second plurality of filters transmits reflected electromagnetic radiation within the second spectral waveband;
wherein the first spectral waveband is different from the second spectral waveband.

2. The system of claim 1, further comprising an endoscope tube, wherein each of the first image sensor and the second image sensor is disposed within an interior cavity of the endoscope tube.

3. The system of claim 1, wherein the first plurality of filters of the first multispectral filter array comprises:
a first plurality of red filters that transmit red electromagnetic radiation;
a first plurality of green filters that transmit green electromagnetic radiation;
a first plurality of blue filters that transmit blue electromagnetic radiation; and
a first plurality of spectral filters that transmit the reflected electromagnetic radiation within the first spectral waveband; and
wherein the second plurality of filters of the second multispectral filter array comprises:
a second plurality of red filters that transmit the red electromagnetic radiation;
a second plurality of green filters that transmit the green electromagnetic radiation;
a second plurality of blue filters that transmit the blue electromagnetic radiation; and
a second plurality of spectral filters that transmit the reflected electromagnetic radiation within the second spectral waveband.

4. The system of claim 3, wherein at least one of the first plurality of filters of the first multispectral filter array or the second plurality of filters of the second multispectral filter array comprises a third plurality of spectral filters that transmit reflected electromagnetic radiation within a third waveband, and wherein the third waveband is different from the first spectral waveband or the second spectral waveband.

5. The system of claim 3, wherein one or more of:
a quantity of the first plurality of green filters is greater than a quantity of either of the first plurality of red filters or the first plurality of blue filters; or
a quantity of the second plurality of green filters is greater than a quantity of either of the second plurality of red filters or the second plurality of blue filters.

6. The system of claim 1, wherein the emitter further comprises a third spectral source that emits electromagnetic radiation within a third spectral waveband, and wherein the third spectral waveband is different from the first spectral waveband or the second spectral waveband; and
wherein the first plurality of filters of the first multispectral filter array comprises:
a first plurality of spectral filters that transmits the reflected electromagnetic radiation within the first spectral waveband; and
a third plurality of spectral filters that transmits reflected electromagnetic radiation within the third spectral waveband.

7. The system of claim 6, wherein the emitter further comprises a fourth spectral source that emits electromagnetic radiation within a fourth spectral waveband, and wherein the fourth spectral waveband is different from each of the first spectral waveband, the second spectral waveband, and the third spectral waveband; and
wherein the second plurality of filters of the second multispectral filter array comprises:
a second plurality of spectral filters that transmits the reflected electromagnetic radiation within the second spectral waveband; and
a fourth plurality of spectral filters that transmits reflected electromagnetic radiation within the fourth spectral waveband.

8. The system of claim 1, wherein the first multispectral filter array and the second multispectral filter array collectively transmit a plurality of spectral wavebands of electromagnetic radiation selected for multispectral visualization or fluorescence visualization of a scene.

9. The system of claim 8, wherein each of the plurality of spectral wavebands comprises electromagnetic radiation corresponding with a spectral reflectance waveband of a tissue;
wherein the spectral reflectance waveband of the tissue comprises one or more wavelengths of electromagnetic radiation that the tissue reflects; and
wherein the tissue comprises one or more of venous tissue, arterial tissue, ureter tissue, nervous tissue, cardiovascular tissue, or cancerous tissue.

10. The system of claim 1, wherein each of the first spectral waveband and the second spectral waveband is a narrowband of wavelengths selected for multispectral visualization or fluorescence visualization of a scene.

11. The system of claim 10, wherein each of the first spectral waveband and the second spectral waveband is 20 nm wide or less.

12. The system of claim 1, wherein at least one of the first spectral waveband or the second spectral waveband is within a near infrared waveband of the electromagnetic spectrum.

13. The system of claim 1, wherein at least one of the first spectral waveband or the second spectral waveband is within a visible waveband of the electromagnetic spectrum and is 20 nm wide or less.

14. The system of claim 1, wherein at least one of the first multispectral filter array or the second multispectral filter array comprises a tunable filter.

15. The system of claim 1, wherein the first image sensor outputs a first data frame simultaneously with the second image sensor outputting a second data frame;
wherein each of the first data frame and the second data frame comprises color imaging data;
wherein the first data frame comprises first spectral imaging data associated with the first spectral waveband; and
wherein the second data frame comprises second spectral imaging data associated with the second spectral waveband.

16. The system of claim 15, wherein at least one of the first spectral imaging data or the second spectral imaging data comprises pixel integration values for pixels accumulating a fluorescence relaxation emission by one or more of a fluorescent reagent or an auto fluorescing tissue.

17. The system of claim 15, wherein at least one of the first spectral imaging data or the second spectral imaging data comprises pixel integration values for pixels accumulating a spectral reflectance that is reflected by one or more of a tissue structure, a chemical process, or a biological process.

18. The system of claim 15, further comprising an image signal processor in communication with the first image sensor and the second image sensor, wherein the image signal processor is configured to execute instructions comprising:
calculating dimensional information based on pixel integration values for the first data frame and the second data frame, and further based on a relative position of the first pixel array and the second pixel array.

19. The system of claim 18, wherein the instructions executed by the image signal processor further comprise rendering a three-dimensional image of a scene based on the dimensional information.

20. The system of claim 18, wherein the instructions executed by the image signal processor further comprises generating an overlay frame comprising:
the color imaging data; and
a false color overlay rendered based on one or more of the first spectral imaging data or the second spectral imaging data;
wherein the false color overlay highlights a location of one or more of a tissue structure, a chemical process, or a biological process within a scene.
